(19) United States
(12) Patent Application Publication
(10) Pub. No.: US 2016/0326589 A1
(43) Pub. Date: Nov. 10, 2016

(54) METHOD OF DIAGNOSING PATIENTS WITH CONDITIONS CAUSED BY MENDELIAN MUTATION

(71) Applicant: SULTAN TURKI AL-SEDAIRY, RIYADH (SA)

(72) Inventor: SULTAN TURKI AL-SEDAIRY, RIYADH (SA)

(21) Appl. No.: 15/146,867

(22) Filed: May 4, 2016

Related U.S. Application Data
(60) Provisional application No. 62/156,872, filed on May 4, 2015.

(51) Int. Cl. C12Q 1/68

(52) U.S. Cl. CPC C12Q 1/6883; C12Q 2600/156

(57) ABSTRACT

The method of diagnosing patients with conditions caused by Mendelian mutations is a genetic panel-based diagnostic method for determining if a patient has a condition (or a proclivity for a condition) based on detection of one or more specific genetic markers. A sample is first obtained from a patient and the sample is assayed to determine the presence of at least one genetic marker. The assay is a sequencing-based multiplexing assay designed for the detection of specific Mendelian mutations. The patient is then diagnosed with a particular condition (or with a proclivity for that condition) if the at least one genetic marker is detected.

### CROSS-REFERENCE TO RELATED APPLICATION

This application claims the benefit of U.S. Provisional Patent Application Ser. No. 62/156,872, filed on May 4, 2015, and which is hereby incorporated by reference in its entirety.

### BACKGROUND OF THE INVENTION

1. Field of the Invention

The present invention relates to genetic detection of conditions, and particularly to a method for diagnosing patients with conditions caused by Mendelian mutations, or diagnosing such patients as having a proclivity towards developing such conditions.

2. Description of the Related Art

Genomics have ushered in a new era for clinical medicine. The ability to scan the entire genome (or its coding part) for disease causing mutations relatively free of clinical bias has uncovered the limited sensitivity and specificity of making diagnoses on clinical grounds only. This was first apparent with the advent of array-CGH that specifically targets large genomic mutations. Subsequently, whole genome sequencing (WGS) and whole exome sequencing (WES) confirmed the same pattern. This raises the interesting question of whether all patients with a suspected genetic diagnosis should have WGS/WES as the initial diagnostic test. Pending data on the validity of this approach, one has to consider some practical challenges. Cost remains a significant hurdle that prevents most patients, especially in less wealthy countries, from accessing WGS/WES. While the running cost will continue to decrease, the challenge of identifying a single causal variant from among tens of thousands will remain formidable for the foreseeable future. In addition, debate still rages over the issue of incidental findings with changing guidelines reflective of the strong and sound argument made by camps on either side of the debate, especially in pediatrics. Gene panels that specifically target a disease relevant to the patient's presentation appear to address some of these limitations but suffer from lack of uniformity in design and are typically too focused on a particular phenotype that they may miss atypical presentation. This is a particular issue when it comes to Mendelian mutations, which are single-gene mutations which may result in a wide variety of disorders. It would obviously be desirable to be able to develop an assay that addresses these limitations. Thus, a method of diagnosing patients with conditions caused by Mendelian mutations solving the aforementioned problems is desired.

### SUMMARY OF THE INVENTION

The method of diagnosing patients with conditions caused by Mendelian mutations is a genetic panel-based diagnostic method for determining if a patient has a condition (or a proclivity for a condition) based on detection of one or more specific genetic markers. A sample is first obtained from a patient and the sample is assayed to determine the presence of at least one genetic marker. The assay is a sequencing-based multiplexing assay designed for the detection of specific Mendelian mutations (the set of which are referred to herein as the “Mendeliome”). The patient is then diagnosed with a particular condition (or with a proclivity for that condition) if the at least one genetic marker is detected.

For detection of cardiovascular disease (or the proclivity therefor), the at least one genetic marker is selected from the group consisting of TTR, MYPN, TTN, COL4A3, KCNH2, SMAD4, NOTCH1, ANK2, PKP2, LDB3, MYH6, MYBPC3, SCN5A, MYL3, CACNA1C, DMD, BAG3, EHMT1, DSG2, ABCC9, KCNE2, RYR2, TTN, TTN-AS1, VCL, SOS1, ANKRD1, ACTN2, DSP, FBN1, CHD7 and combinations thereof.

For detection of deafness (or the proclivity therefor), the at least one genetic marker is selected from the group consisting of UBIAD1, LARS2, GJB2, HGF, MYO6, PCDH15, TMC1, MARVELD2, CDH23, OTOF, LRTOMT, LOXHD1, EDN3, MYO15A, SLC26A4, CLDN14, MARVELD2, WFS1, POU4F3, PTPRQ, SCARF2, COL4A4, USH2A, MYO7A and combinations thereof.

For detection of dermatological conditions (or the proclivity therefor), the at least one genetic marker is selected from the group consisting of XPC, COL7A1, ALDH3A2, SLC39A4, CTSC, ITGB4, TGM1, HPS1, TYR, LAMB3, EOGT, DOCK6, LAMC2, GORAB, KRT5, KRT83, COL18A1, ALDH18A1, FERMT1, EOGT, DCAF17, DSP, NF1 and combinations thereof.

For detection of dysmorphia-dysplasia (or the proclivity therefor), the at least one genetic marker is selected from the group consisting of LIFR, TCOF1, LARP7, EVC, POC1A, HGSNAT, COL2A1, CRTAP, COL11A2, DYM, COL1A1, CREBBP, COL11A1, PYCR1, NIPBL, ROR2, EXT1, ACTB, ADAMTSL2, NEK1, DYNC2H1, IRF6, NSD1, UBE3B, DLL3, EP300, SGSH, EZH2, CHRNG, GALNS, MGAT2, TNFRSF11B, LMNA, ERCC8, CANT1, MMP2, FKBP10, CUL7, GNPAT, FGFR2, FGFR3, MASP1, FREM1, HSPG2, MEOX1, OBSL1, WNT1, COL1A2, COL1A1, ANTXR2, PEX13, ECEL1, KMT2A, KMT2D, PCNT, EBP, UBR1, WISP3, DLX5, IFT122, HRAS, SERPINF1, RIPK4, LEPRE1, BRAF, NFIX, FBN1, NF1, TMEM67, COLEC11, SCARF2 and combinations thereof.

For detection of endocrine conditions (or the proclivity therefor), the at least one genetic marker is selected from the group consisting of TBCE, GHR, GHRHR, BBS5, SHOX and combinations thereof.

For detection of gastrointestinal conditions (or the proclivity therefor), the at least one genetic marker is selected from the group consisting of UGT1A1, UGT1A10, UGT1A3, UGT1A4, UGT1A5, UGT1A6, UGT1A7, UGT1A8, UGT1A9, JAG1, BAAT, ATP7B, TJP2, EPCAM, ABCB4, ABCC2, LRBA, SLC10A2, ABCB11, VIPAS39, FAH, G6PC and combinations thereof.

For detection of hematological conditions (or the proclivity therefor), the at least one genetic marker is selected from the group consisting of BLM, FANCA, FANCM, BRCA2, ASXL1 and combinations thereof.

For detection of inborn errors of metabolism (or the proclivity therefor), the at least one genetic marker is selected from the group consisting of L2HGDH, MCCC2, SLC37A4, ARSB, HSD3B7, DBT, PHKG2, BTD, MUT, ASL, DPAGT1, ASAH1, AMT, BCKDHB, BCKDHA, CBS, PAH, CLN8, GBA, ACADM, SLC3A1, MMACHC, PTS, GNS, GCDH, SLC22A5, GAA, MMADHC, PYGL, ASS1, CPS1, H6PD, PTS, PGM1, IVD, ARG1, ASAH1, GLB1, OXCT1, OPLAH, FAH, G6PC, PEX1 and combinations thereof.

For detection of neurological disorders (or the proclivity therefor), the at least one genetic marker is selected from the group consisting of L1CAM, ABCD1, DYSF, GBA2, TRAPPC9, CYP2U1, PANK2, ARL13B, KIF7, ERLIN2, PSAP, VAPB, FKTN, PLP1, GDAP1, ASPM, LAMA2, MECP2, CDK5RAP2, WDR81, ABAT, NDE1, WDR45B, H5D17B4, HEXA, SPG11, PDGFRB, HUWE1, SLC25A19, ARHGEF6, ADRA2B, RELN, CENPJ, ARL14EP, PHGDH, ARID1B, WNK1, SEPN1, RNASEH2C, RNASEH2B, CYP27A1, ATN1, AHI1, STXBP1, CDKL5, MED23, ISPD, CEP57, AGRN, FKRP, ADCK3, SCN2A, MFSD8, TYMP, FLVCR2, SPG20, CACNA1G, PLA2G6, CLN6, WDR62, PEX26, KIF1A, PNPO, LARGE, YARS, KIAA0196, CCDC88C, OPTN, OCLN, ATRX, ATL1, GNE, PEX12, SPTBN2, PEX16, COL6A1, COL6A3, COL6A2, HEPACAM, LRPPRC, RYR1, NTRK1, CAPN3, SOD1, COG6, ATP2B3, DPYD, TUBA1A, TCTN1, CPA6, ABHD12, NPC2, MPDZ, SYNGAP1, PEX5, PEX6, POMT1, POMT2, MCPH1, CASC5, SGCB, SGCA, POMGNT2, TRMT1, ARFGEF2, SYNE2, ADK, ZNF526, FOXG1, ALS2, C5orf42, TMEM237, C12orf57, TMEM67, PEX1 and combinations thereof.

For detection of pelvic inflammatory disease (or the proclivity therefor), the at least one genetic marker is selected from the group consisting of IL7R, JAK3, CD40LG, AK2, DCLRE1C, CD40, AICDA, MLPH, NHEJ1, RAB27A, RAG2, RAG1, BTK, ATM, LYST, CYBB, AIRE, DOCK8, SLC17A5, STATS, WAS, CD247, DNMT3B, FLG, NCF2, ADA, RFXANK, PTPRC, COLEC11 and combinations thereof.

For detection of pulmonary conditions (or the proclivity therefor), the at least one genetic marker is selected from the group consisting of SFTPB, CFTR and combinations thereof.

For detection of renal conditions (or the proclivity therefor), the at least one genetic marker is selected from the group consisting of IQCB1, COL4A6, NPHP3, SLC4A4, DDX39A, SMARCAL1, PKHD1, LAMB2, NEK8, NPHP4, FRAS1, XDH, MKS1, FAN1, TCTN2, NPHS1, CC2D2A, TMEM231, UPK3A, CEP290, NPHP4, COL4A4, TMEM67, C5orf42, TMEM237 and combinations thereof.

For detection of vision disorders (or the proclivity therefor), the at least one genetic marker is selected from the group consisting of ALMS1, CRB1, CHST6, CRYBA1, PRSS56, GUCY2D, SNRNP200, PDE6C, CNGA3, C8orf37, ABCA4, BBS10, CERKL, GPR125, NHS, LTBP2, GCNT2, RLBP1, MIP, RP1L1, CHM, EYS, TULP1, IGFBP7, CYP1B1, LRAT, MERTK, CNNM4, RP1, RP2, LCA5, MFRP, CNGB1, CACNA1F, KCNV2, CRX, PROM1, TRPM1, PAX6, IMPG2, CDHR1, GPR179, CRYGC, CRYGD, NMNAT1, GALT, ARL6, LRP5, WDR19, SLC4A11, GDF3, SLC16A12, RGS9, RDH12, ADAMS, AIPL1, FAM161A, RPGRIP1, RAB3GAP2, RAB3GAP1, EFEMP1, BEST1, RPE65, EPHA2, FZD4, PRPH2, CRYAA, KCNJ13, NR2E3, BBS9, BBS1, BBS2, BBS5, BBS4, BBS7, SPATA7, CHD7, USH2A, MYO7A, C12orf57, CEP290, NPHP4 and combinations thereof.

These and other features of the present invention will become readily apparent upon further review of the following specification.

### DETAILED DESCRIPTION OF THE PREFERRED EMBODIMENTS

The method of diagnosing patients with conditions caused by Mendelian mutations is a genetic panel-based diagnostic method for determining if a patient has a condition (or a proclivity for a condition) based on detection of one or more specific genetic markers. A sample is first obtained from a patient and the sample is assayed to determine the presence of at least one genetic marker. The assay is a sequencing-based multiplexing assay designed for the detection of specific Mendelian mutations (the set of which are referred to herein as the “Mendeliome”). The patient is then diagnosed with a particular condition (or with a proclivity for that condition) if the at least one genetic marker is detected.

For detection of cardiovascular disease (or the proclivity therefor), the at least one genetic marker is selected from the group consisting of TTR, MYPN, TTN, COL4A3, KCNH2, SMAD4, NOTCH1, ANK2, PKP2, LDB3, MYH6, MYBPC3, SCN5A, MYL3, CACNA1C, DMD, BAG3, EHMT1, DSG2, ABCC9, KCNE2, RYR2, TTN, TTN-AS1, VCL, SOS1, ANKRD1, ACTN2, DSP, FBN1, CHD7 and combinations thereof. The details of the cardiovascular panel are given below in Table 1.

TABLE 1
Cardiovascular Panel

| | | | | | Number | |
|---|---|---|---|---|---|---|
| Gene | Chr | Start | End | Transcript | of Exons | MIM |
| TTR | chr18 | 29171729 | 29178986 | NM_000371 | 4 | 176300 |
| MYPN | chr10 | 69869189 | 69971773 | NM_001256268 | 24 | 608517 |
| TTN | chr2 | 179390716 | 179672150 | NM_001267550 | 363 | 188840 |
| COL4A3 | chr2 | 228029280 | 228179508 | NM_000091 | 52 | 120070 |
| KCNH2 | chr7 | 150642043 | 150675402 | NM_000238 | 15 | 152427 |
| SMAD4 | chr18 | 48556582 | 48611411 | NM_005359 | 12 | 600993 |
| NOTCH1 | chr9 | 139388895 | 139440238 | NM_017617 | 34 | 190198 |
| ANK2 | chr4 | 113970784 | 114304896 | NM_001148 | 46 | 106410 |
| PKP2 | chr12 | 32943679 | 33049780 | NM_004572 | 14 | 602861 |
| LDB3 | chr10 | 88428205 | 88495824 | NM_001171610 | 14 | 605906 |
| MYH6 | chr14 | 23851198 | 23877486 | NM_002471 | 39 | 160710 |
| MYBPC3 | chr11 | 47352956 | 47374253 | NM_000256 | 34 | 600958 |
| SCN5A | chr3 | 38589552 | 38691163 | NM_198056 | 28 | 600163 |
| MYL3 | chr3 | 46899356 | 46904973 | NM_000258 | 7 | 160790 |
| CACNA1C | chr12 | 2162415 | 2807115 | NM_199460 | 50 | 114205 |
| DMD | chrX | 31137344 | 33038317 | NM_004007 | 78 | 300377 |
| BAG3 | chr10 | 121410881 | 121437329 | NM_004281 | 4 | 603883 |
| EHMT1 | chr9 | 140513443 | 140730578 | NM_024757 | 27 | 607001 |
| DSG2 | chr18 | 29078026 | 29128814 | NM_001943 | 15 | 125671 |
| ABCC9 | chr12 | 21958107 | 22089628 | NM_005691 | 38 | 601439 |
| KCNE2 | chr21 | 35736322 | 35743440 | NM_172201 | 2 | 603796 |
| RYR2 | chr1 | 237205701 | 237997288 | NM_001035 | 105 | 180902 |
| TTN | chr2 | 179390716 | 179672150 | NM_001267550 | 363 | 188840 |
| TTN-AS1 | chr2 | 179385910 | 179644690 | NR_038271 | 7 | NA |
| VCL | chr10 | 75757871 | 75879914 | NM_014000 | 22 | 193065 |
| SOS1 | chr2 | 39208689 | 39347604 | NM_005633 | 23 | 182530 |
| ANKRD1 | chr10 | 92671856 | 92681032 | NM_014391 | 9 | 609599 |
| ACTN2 | chr1 | 236849753 | 236927927 | NM_001278344 | 23 | 102573 |

For detection of deafness (or the proclivity therefor), the at least one genetic marker is selected from the group consisting of UBIAD1, LARS2, GJB2, HGF, MYO6, PCDH15, TMC1, MARVELD2, CDH23, OTOF, LRTOMT, LOXHD1, EDN3, MYO15A, SLC26A4, CLDN14, MARVELD2, WFS1, POU4F3, PTPRQ, SCARF2, COL4A4, USH2A, MYO7A and combinations thereof. The details of the deafness panel are given below in Table 2.

TABLE 2
Deafness Panel

| | | | | | Number | |
|---|---|---|---|---|---|---|
| | | | | | of | |
| Gene | Chr | Start | End | Transcript | Exons | MIM |
| UBIAD1 | chr1 | 11333254 | 11348491 | NM_013319 | 2 | 611632 |
| LARS2 | chr3 | 45430074 | 45590328 | NM_015340 | 22 | 604544 |
| GJB2 | chr13 | 20761603 | 20767114 | NM_004004 | 2 | 121011 |
| HGF | chr7 | 81331443 | 81399452 | NM_000601 | 18 | 142409 |
| MYO6 | chr6 | 76458908 | 76629254 | NM_004999 | 35 | 600970 |
| PCDH15 | chr10 | 55568451 | 56561051 | NM_001142769 | 36 | 605514 |
| TMC1 | chr9 | 75136716 | 75451267 | NM_138691 | 24 | 606706 |
| MARVELD2 | chr5 | 68710938 | 68737890 | NM_001038603 | 7 | 610572 |
| CDH23 | chr10 | 73156690 | 73575704 | NM_022124 | 68 | 605516 |
| OTOF | chr2 | 26680070 | 26781566 | NM_194248 | 47 | 603681 |
| LRTOMT | chr11 | 71791376 | 71821828 | NM_001145309 | 9 | 612414 |
| LOXHD1 | chr18 | 44057216 | 44236996 | NM_144612 | 40 | 613072 |
| EDN3 | chr20 | 57875498 | 57901047 | NM_000114 | 6 | 131242 |
| MYO15A | chr17 | 18012019 | 18083116 | NM_016239 | 65 | 602666 |
| SLC26A4 | chr7 | 107301079 | 107358252 | NM_000441 | 21 | 605646 |
| CLDN14 | chr21 | 37832919 | 37948867 | NM_001146077 | 3 | 605608 |
| MARVELD2 | chr5 | 68710938 | 68737890 | NM_001038603 | 7 | 610572 |
| WFS1 | chr4 | 6271576 | 6304992 | NM_001145853 | 8 | 606201 |
| POU4F3 | chr5 | 145718586 | 145720083 | NM_002700 | 2 | 602460 |
| PTPRQ | chr12 | 80838125 | 81073968 | NM_001145026 | 42 | 603317 |
| SCARF2 | chr22 | 20778873 | 20792146 | NM_182895 | 11 | 613619 |
| COL4A4 | chr2 | 227867426 | 228029275 | NM_000092 | 48 | 120131 |
| USH2A | chr1 | 215796235 | 216596738 | NM_206933 | 72 | 608400 |
| MYO7A | chr11 | 76839309 | 76926286 | NM_000260 | 49 | 276903 |

For detection of dermatological conditions (or the proclivity therefor), the at least one genetic marker is selected from the group consisting of XPC, COL7A1, ALDH3A2, SLC39A4, CTSC, ITGB4, TGM1, HPS1, TYR, LAMBS, EOGT, DOCK6, LAMC2, GORAB, KRT5, KRT83, COL18A1, ALDH18A1, FERMT1, EOGT, DCAF17, DSP, NF1 and combinations thereof. The details of the dermatological panel are given below in Table 3.

TABLE 3
Dermatological Panel

| | | | | | Number | |
|---|---|---|---|---|---|---|
| | | | | | of | |
| Gene | Chr | Start | End | Transcript | Exons | MIM |
| XPC | chr3 | 14186647 | 14220172 | NM_001145769 | 16 | 613208 |
| COL7A1 | chr3 | 48601505 | 48632593 | NM_000094 | 118 | 120120 |
| ALDH3A2 | chr17 | 19552063 | 19580904 | NM_001031806 | 11 | 609523 |
| SLC39A4 | chr8 | 145637797 | 145642273 | NM_130849 | 12 | 607059 |
| CTSC | chr11 | 88026759 | 88070941 | NM_001814 | 7 | 602365 |
| ITGB4 | chr17 | 73717515 | 73753899 | NM_000213 | 40 | 147557 |
| TGM1 | chr14 | 24718319 | 24732416 | NM_000359 | 15 | 190195 |
| HPS1 | chr10 | 100188902 | 100206704 | NM_182639 | 10 | 604982 |
| TYR | chr11 | 88911039 | 89028927 | NM_000372 | 5 | 606933 |
| LAMB3 | chr1 | 209788217 | 209825674 | NM_001127641 | 23 | 150310 |
| EOGT | chr3 | 69024365 | 69063112 | NM_001278689 | 18 | 614789 |
| DOCK6 | chr19 | 11309968 | 11373168 | NM_020812 | 48 | 614194 |
| LAMC2 | chr1 | 183155173 | 183210406 | NM_018891 | 22 | 150292 |
| GORAB | chr1 | 170501262 | 170522974 | NR_027397 | 5 | 607983 |
| KRT5 | chr12 | 52908358 | 52914243 | NM_000424 | 9 | 148040 |
| KRT83 | chr12 | 52708084 | 52715182 | NM_002282 | 9 | 602765 |
| COL18A1 | chr21 | 46875423 | 46933634 | NM_030582 | 42 | 120328 |
| ALDH18A1 | chr10 | 97365685 | 97416567 | NM_001017423 | 18 | 138250 |
| FERMT1 | chr20 | 6055491 | 6104191 | NM_017671 | 15 | 607900 |
| EOGT | chr3 | 69024365 | 69063112 | NM_001278689 | 18 | 614789 |
| DCAF17 | chr2 | 172290760 | 172341562 | NM_025000 | 14 | 612515 |
| DSP | chr6 | 7541869 | 7586946 | NM_004415 | 24 | 125647 |

For detection of dysmorphia-dysplasia (DD) (or the proclivity therefor), the at least one genetic marker is selected from the group consisting of LIFR, TCOF1, LARP7, EVC, POC1A, HGSNAT, COL2A1, CRTAP, COL11A2, DYM, COL1A1, CREBBP, COL11A1, PYCR1, NIPBL, ROR2, EXT1, ACTB, ADAMTSL2, NEK1, DYNC2H1, IRF6, NSD1, UBE3B, DLL3, EP300, SGSH, EZH2, CHRNG, GALNS, MGAT2, TNFRSF11B, LMNA, ERCC8, CANT1, MMP2, FKBP10, CUL7, GNPAT, FGFR2, FGFR3, MASP1, FREM1, HSPG2, MEOX1, OBSL1, WNT1, COL1A2, COL1A1, ANTXR2, PEX13, ECEL1, KMT2A, KMT2D, PCNT, EBP, UBR1, WISP3, DLX5, IFT122, HRAS, SERPINF1, RIPK4, LEPRE1, BRAF, NFIX, FBN1, NF1, TMEM67, COLEC11, SCARF2 and combinations thereof. The details of the dysmorphia-dysplasia panel are given below in Table 4.

TABLE 4
Dysmorphia-Dysplasia (DD) Panel

| | | | | | Number | |
|---|---|---|---|---|---|---|
| | | | | | of | |
| Gene | Chr | Start | End | Transcript | Exons | MIM |
| LIFR | chr5 | 38475064 | 38595507 | NM_002310 | 20 | 151443 |
| TCOF1 | chr5 | 149737201 | 149779871 | NM_001135243 | 27 | 606847 |
| LARP7 | chr4 | 113558119 | 113578748 | NM_001267039 | 15 | 612026 |
| EVC | chr4 | 5712923 | 5816031 | NM_153717 | 21 | 604831 |
| POC1A | chr3 | 52109248 | 52188706 | NM_015426 | 11 | 614783 |
| HGSNAT | chr8 | 42995591 | 43057970 | NM_152419 | 18 | 610453 |
| COL2A1 | chr12 | 48366747 | 48398285 | NM_001844 | 54 | 120140 |
| CRTAP | chr3 | 33155449 | 33189265 | NM_006371 | 7 | 605497 |
| COL11A2 | chr6 | 4610635 | 4637414 | NM_080680 | 65 | 120290 |
| DYM | chr18 | 46570171 | 46987079 | NM_017653 | 17 | 607461 |
| COL1A1 | chr17 | 48261456 | 48279000 | NM_000088 | 51 | 120150 |
| CREBBP | chr16 | 3775055 | 3930121 | NM_004380 | 31 | 600140 |
| COL11A1 | chr1 | 103342022 | 103574052 | NM_080629 | 67 | 120280 |
| PYCR1 | chr17 | 79890266 | 79894968 | NM_153824 | 8 | 179035 |
| NIPBL | chr5 | 36876860 | 37065921 | NM_133433 | 47 | 608667 |
| ROR2 | chr9 | 94484877 | 94712444 | NM_004560 | 9 | 602337 |
| EXT1 | chr8 | 118811601 | 119124058 | NM_000127 | 11 | 608177 |
| ACTB | chr7 | 5566778 | 5570232 | NM_001101 | 6 | 102630 |
| ADAMTSL2 | chr9 | 136399974 | 136440641 | NM_014694 | 19 | 612277 |
| NEK1 | chr4 | 170314420 | 170533778 | NM_001199397 | 36 | 604588 |
| DYNC2H1 | chr11 | 102980159 | 103350591 | NM_001080463 | 90 | 603297 |
| IRF6 | chr1 | 209958967 | 209979520 | NM_006147 | 9 | 607199 |
| NSD1 | chr5 | 176560832 | 176727214 | NM_022455 | 23 | 606681 |
| UBE3B | chr12 | 109915427 | 109974510 | NM_183415 | 28 | 608047 |
| DLL3 | chr19 | 39989556 | 39999121 | NM_203486 | 9 | 602768 |
| EP300 | chr22 | 41488613 | 41576081 | NM_001429 | 31 | 602700 |
| SGSH | chr17 | 78183078 | 78194199 | NM_000199 | 8 | 605270 |
| EZH2 | chr7 | 148504463 | 148581441 | NM_001203247 | 20 | 601573 |
| CHRNG | chr2 | 233404436 | 233411038 | NM_005199 | 12 | 100730 |
| GALNS | chr16 | 88880141 | 88923374 | NM_000512 | 14 | 612222 |
| MGAT2 | chr14 | 50087488 | 50090199 | NM_002408 | 1 | 602616 |
| TNFRSF11B | chr8 | 119935795 | 119964383 | NM_002546 | 5 | 602643 |
| LMNA | chr1 | 156095950 | 156109880 | NM_001257374 | 13 | 150330 |
| ERCC8 | chr5 | 60169658 | 60240905 | NM_000082 | 12 | 609412 |
| CANT1 | chr17 | 76987797 | 77005899 | NM_001159773 | 5 | 613165 |
| MMP2 | chr16 | 55513080 | 55540586 | NM_004530 | 13 | 120360 |
| FKBP10 | chr17 | 39968961 | 39979469 | NM_021939 | 10 | 607063 |
| CUL7 | chr6 | 43005354 | 43021683 | NM_001168370 | 26 | 609577 |
| GNPAT | chr1 | 231376918 | 231413719 | NM_014236 | 16 | 602744 |
| FGFR2 | chr10 | 123241366 | 123353481 | NM_001144913 | 17 | 176943 |
| FGFR3 | chr4 | 1795038 | 1810599 | NM_001163213 | 18 | 134934 |
| MASP1 | chr3 | 186933872 | 187009810 | NM_001879 | 16 | 600521 |
| FREM1 | chr9 | 14734663 | 14910993 | NM_144966 | 38 | 608944 |
| HSPG2 | chr1 | 22148736 | 22263750 | NM_005529 | 97 | 142461 |
| MEOX1 | chr17 | 41717757 | 41738931 | NM_004527 | 3 | 600147 |
| OBSL1 | chr2 | 220415449 | 220436268 | NM_015311 | 21 | 610991 |
| WNT1 | chr12 | 49372235 | 49376396 | NM_005430 | 4 | 164820 |
| COL1A2 | chr7 | 94023872 | 94060544 | NM_000089 | 52 | 120160 |
| COL1A1 | chr17 | 48261456 | 48279000 | NM_000088 | 51 | 120150 |
| ANTXR2 | chr4 | 80898661 | 80994477 | NM_001145794 | 16 | 608041 |
| PEX13 | chr2 | 61244811 | 61279125 | NM_002618 | 4 | 601789 |
| ECEL1 | chr2 | 233344536 | 233352532 | NM_004826 | 18 | 605896 |
| KMT2A | 11 | 118307205 | 118397539 | NM_001197104 | 36 | 159555 |
| KMT2D | chr12 | 49412758 | 49453557 | NM_003482 | 54 | 602113 |
| PCNT | chr21 | 47744035 | 47865682 | NM_006031 | 47 | 605925 |
| EBP | chrX | 48380163 | 48387104 | NM_006579 | 5 | 300205 |
| UBR1 | chr15 | 43235097 | 43398286 | NM_174916 | 47 | 605981 |
| WISP3 | chr6 | 112375277 | 112390887 | NM_003880 | 6 | 603400 |
| DLX5 | chr7 | 96649701 | 96654143 | NM_005221 | 3 | 600028 |
| IFT122 | chr3 | 129158967 | 129239191 | NM_052985 | 31 | 606045 |
| HRAS | chr11 | 532241 | 535550 | NM_005343 | 6 | 190020 |
| SERPINF1 | chr17 | 1665258 | 1680859 | NM_002615 | 8 | 172860 |
| RIPK4 | chr21 | 43159528 | 43187249 | NM_020639 | 8 | 605706 |
| LEPRE1 | chr1 | 43212005 | 43232755 | NM_022356 | 15 | 610339 |
| BRAF | chr7 | 140433812 | 140624564 | NM_004333 | 18 | 164757 |
| NFIX | chr19 | 13135394 | 13209610 | NM_001271043 | 11 | 164005 |
| FBN1 | chr15 | 48700502 | 48937985 | NM_000138 | 66 | 134797 |
| NF1 | chr17 | 29421944 | 29704695 | NM_001042492 | 58 | 613113 |
| TMEM67 | chr8 | 94767071 | 94831460 | NR_024522 | 29 | 609884 |
| COLEC11 | chr2 | 3642421 | 3692234 | NM_199235 | 8 | 612502 |

For detection of endocrine conditions (or the proclivity therefor), the at least one genetic marker is selected from the group consisting of TBCE, GHR, GHRHR, BBS5, SHOX and combinations thereof. The details of the endocrine panel are given below in Table 5.

TABLE 5
Endocrine Panel

| | | | | | Number | |
|---|---|---|---|---|---|---|
| | | | | | of | |
| Gene | Chr | Start | End | Transcript | Exons | MIM |
| TBCE | chr1 | 235530727 | 235612280 | NM_003193 | 17 | 604934 |
| GHR | chr5 | 42424553 | 42721980 | NM_001242399 | 10 | 600946 |
| GHRHR | chr7 | 31003635 | 31019146 | NM_000823 | 13 | 139191 |
| BBS5 | chr2 | 170336005 | 170363165 | NM_152384 | 12 | 603650 |
| SHOX | chrX | 535078 | 570146 | NM_006883 | 6 | 400020 |

For detection of gastrointestinal (GI) conditions (or the proclivity therefor), the at least one genetic marker is selected from the group consisting of UGT1A1, UGT1A10, UGT1A3, UGT1A4, UGT1A5, UGT1A6, UGT1A7, UGT1A8, UGT1A9, JAG1, BAAT, ATP7B, TJP2, EPCAM, ABCB4, ABCC2, LRBA, SLC10A2, ABCB11, VIPAS39, FAH, G6PC and combinations thereof. The details of the gastrointestinal panel are given below in Table 6.

TABLE 6
Gastrointestinal (GI) Panel

| | | | | | Number | |
|---|---|---|---|---|---|---|
| | | | | | of | |
| Gene | Chr | Start | End | Transcript | Exons | MIM |
| UGT1A1 | chr2 | 234668894 | 234681945 | NM_000463 | 5 | 191740 |
| UGT1A10 | chr2 | 234545100 | 234681951 | NM_019075 | 5 | 606435 |
| UGT1A3 | chr2 | 234637754 | 234681945 | NM_019093 | 5 | 606428 |
| UGT1A4 | chr2 | 234627437 | 234681945 | NM_007120 | 5 | 606429 |
| UGT1A5 | chr2 | 234621638 | 234681945 | NM_019078 | 5 | 606430 |
| UGT1A6 | chr2 | 234600253 | 234681946 | NM_001072 | 5 | 606431 |
| UGT1A7 | chr2 | 234601511 | 234681951 | NM_001072 | 5 | 606432 |
| UGT1A8 | chr2 | 234526291 | 234681956 | NM_019076 | 5 | 606433 |
| UGT1A9 | chr2 | 234580499 | 234681946 | NM_021027 | 5 | 606434 |
| JAG1 | chr20 | 10618331 | 10654694 | NM_000214 | 26 | 601920 |
| BAAT | chr9 | 104122698 | 104147287 | NM_001701 | 4 | 602938 |
| ATP7B | chr13 | 52506805 | 52585630 | NM_000053 | 21 | 606882 |
| TJP2 | chr9 | 71820077 | 71870124 | NM_001170416 | 23 | 607709 |
| EPCAM | chr2 | 47596286 | 47614167 | NM_002354 | 9 | 185535 |
| ABCB4 | chr7 | 87031360 | 87105019 | NM_018849 | 28 | 171060 |
| ABCC2 | chr10 | 101542462 | 101611662 | NM_000392 | 32 | 601107 |
| LRBA | chr4 | 151185810 | 151936649 | NM_006726 | 58 | 606453 |
| SLC10A2 | chr13 | 103696347 | 103719196 | NM_000452 | 6 | 601295 |
| ABCB11 | chr2 | 169779448 | 169887833 | NM_003742 | 28 | 603201 |
| VIPAS39 | chr14 | 77893018 | 77924295 | NM_022067 | 21 | 613401 |

For detection of hematological conditions (or the proclivity therefor), the at least one genetic marker is selected from the group consisting of BLM, FANCA, FANCM, BRCA2, ASXL1 and combinations thereof. The details of the hematology panel are given below in Table 7.

TABLE 7
Hematology Panel

| | | | | | Num- | |
|---|---|---|---|---|---|---|
| | | | | | ber of | |
| | | | | | Ex- | |
| Gene | Chr | Start | End | Transcript | ons | MIM |
| BLM | chr15 | 91260578 | 91358686 | NM_000057 | 22 | 604610 |
| FANCA | chr16 | 89803958 | 89883065 | NM_000135 | 43 | 607139 |
| FANCM | chr14 | 45605135 | 45670093 | NM_020937 | 23 | 609644 |
| BRCA2 | chr13 | 32889616 | 32973809 | NM_000059 | 27 | 600185 |
| ASXL1 | chr20 | 30946146 | 31027122 | NM_015338 | 12 | 612990 |

For detection of inborn errors of metabolism (IBM) (or the proclivity therefor), the at least one genetic marker is selected from the group consisting of L2HGDH, MCCC2, SLC37A4, ARSB, HSD3B7, DBT, PHKG2, BTD, MUT, ASL, DPAGT1, ASAH1, AMT, BCKDHB, BCKDHA, CBS, PAH, CLN8, GBA, ACADM, SLC3A1, MMACHC, PTS, GNS, GCDH, SLC22A5, GAA, MMADHC, PYGL, ASS1, CPS1, H6PD, PTS, PGM1, IVD, ARG1, ASAH1, GLB1, OXCT1, OPLAH, FAH, G6PC, PEX1 and combinations thereof. The details of the inborn errors of metabolism panel are given below in Table 8.

TABLE 8
Inborn Errors of Metabolism (IEM) Panel

| | | | | | Number | |
|---|---|---|---|---|---|---|
| | | | | | of | |
| Gene | Chr | Start | End | Transcript | Exons | MIM |
| L2HGDH | chr14 | 50709151 | 50778947 | NM_024884 | 10 | 609584 |
| MCCC2 | chr5 | 70883114 | 70954530 | NM_022132 | 17 | 609014 |
| SLC37A4 | chr11 | 118895060 | 118901616 | NM_001164279 | 11 | 602671 |
| ARSB | chr5 | 78111333 | 78281766 | NM_198709 | 8 | 611542 |
| HSD3B7 | chr16 | 30996518 | 31000473 | NM_025193 | 7 | 607764 |
| DBT | chr1 | 100652477 | 100715409 | NM_001918 | 11 | 248610 |
| PHKG2 | chr16 | 30759619 | 30772497 | NM_001172432 | 11 | 172471 |
| BTD | chr3 | 15643254 | 15687325 | NM_000060 | 4 | 609019 |
| MUT | chr6 | 49398072 | 49431041 | NM_000255 | 13 | 609058 |
| ASL | chr7 | 65540775 | 65558329 | NM_000048 | 17 | 608310 |
| DPAGT1 | chr11 | 118967212 | 118972785 | NM_001382 | 9 | 191350 |
| ASAH1 | chr8 | 17913924 | 17942507 | NM_004315 | 14 | 613468 |
| AMT | chr3 | 49454210 | 49460111 | NM_001164712 | 10 | 238310 |
| BCKDHB | chr6 | 80816343 | 81055987 | NM_000056 | 11 | 248611 |
| BCKDHA | chr19 | 41937222 | 41945843 | NM_018035 | 6 | 608348 |
| CBS | chr21 | 44473300 | 44496472 | NM_001178009 | 18 | 613381 |
| PAH | chr12 | 103232103 | 103311381 | NM_000277 | 13 | 612349 |
| CLN8 | chr8 | 1711869 | 1734736 | NM_018941 | 3 | 607837 |
| GBA | chr1 | 155204238 | 155214653 | NM_001005742 | 12 | 606463 |
| ACADM | chr1 | 76190042 | 76229355 | NM_001127328 | 12 | 607008 |
| SLC3A1 | chr2 | 44502596 | 44547962 | NM_000341 | 10 | 104614 |
| MMACHC | chr1 | 45965855 | 45976739 | NM_015506 | 4 | 609831 |
| PTS | chr11 | 112097087 | 112104695 | NM_000317 | 6 | 612719 |
| GNS | chr12 | 65107221 | 65153226 | NM_002076 | 14 | 607664 |
| GCDH | chr19 | 13001942 | 13010813 | NM_013976 | 12 | 608801 |
| SLC22A5 | chr5 | 131705400 | 131731306 | NM_003060 | 10 | 603377 |
| GAA | chr17 | 78075354 | 78093679 | NM_001079804 | 20 | 606800 |
| MMADHC | chr2 | 150426146 | 150444330 | NM_015702 | 8 | 611935 |
| PYGL | chr14 | 51371934 | 51411248 | NM_002863 | 20 | 613741 |
| ASS1 | chr9 | 133320093 | 133376661 | NM_000050 | 16 | 603470 |
| CPS1 | chr2 | 211342405 | 211543831 | NM_001122633 | 39 | 608307 |
| H6PD | chr1 | 9294862 | 9331394 | NM_004285 | 5 | 138090 |
| PTS | chr11 | 112097087 | 112104695 | NM_000317 | 6 | 612719 |
| PGM1 | chr1 | 64088886 | 64125916 | NM_001172818 | 11 | 171900 |
| IVD | chr15 | 40697685 | 40713512 | NM_002225 | 12 | 607036 |
| ARG1 | chr6 | 131894343 | 131905472 | NM_001244438 | 8 | 608313 |
| ASAH1 | chr8 | 17913924 | 17942507 | NM_004315 | 14 | 613468 |
| GLB1 | chr3 | 33038099 | 33138314 | NM_001079811 | 16 | 611458 |
| OXCT1 | chr5 | 41730166 | 41870791 | NM_000436 | 17 | 601424 |
| OPLAH | chr8 | 145106166 | 145115584 | NM_017570 | 28 | 614243 |
| FAH | chr15 | 80445232 | 80478924 | NM_000137 | 14 | 613871 |
| G6PC | chr17 | 41052813 | 41066450 | NM_000151 | 5 | 613742 |
| PEX1 | chr7 | 92116336 | 92157845 | NM_000466 | 24 | 602136 |

For detection of neurological disorders (or the proclivity therefor), the at least one genetic marker is selected from the group consisting of L1CAM, ABCD1, DYSF, GBA2, TRAPPC9, CYP2U1, PANK2, ARL13B, KIF7, ERLIN2, PSAP, VAPB, FKTN, PLP1, GDAP1, ASPM, LAMA2, MECP2, CDK5RAP2, WDR81, ABAT, NDE1, WDR45B, HSD17B4, HEXA, SPG11, PDGFRB, HUWE1, SLC25A19, ARHGEF6, ADRA2B, RELN, CENPJ, ARL14EP, PHGDH, ARID1B, WNK1, SEPN1, RNASEH2C, RNASEH2B, CYP27A1, ATN1, AHI1, STXBP1, CDKL5, MED23, ISPD, CEP57, AGRN, FKRP, ADCK3, SCN2A, MFSD8, TYMP, FLVCR2, SPG20, CACNA1G, PLA2G6, CLN6, WDR62, PEX26, KIF1A, PNPO, LARGE, YARS, KIAA0196, CCDC88C, OPTN, OCLN, ATRX, ATL1, GNE, PEX12, SPTBN2, PEX16, COL6A1, COL6A3, COL6A2, HEPACAM, LRPPRC, RYR1, NTRK1, CAPN3, SOD1, COG6, ATP2B3, DPYD, TUBA1A, TCTN1, CPA6, ABHD12, NPC2, MPDZ, SYNGAP1, PEX5, PEX6, POMT1, POMT2, MCPH1, CASC5, SGCB, SGCA, POMGNT2, TRMT1, ARFGEF2, SYNE2, ADK, ZNF526, FOXG1, ALS2, C5orf42, TMEM237, C12orf57, TMEM67, PEX1 and combinations thereof. The details of the neurological panel are given below in Table 9.

TABLE 9
Neurological Panel

| | | | | | Number | |
|---|---|---|---|---|---|---|
| | | | | | of | |
| Gene | Chr | Start | End | Transcript | Exons | MIM |
| L1CAM | chrX | 153126968 | 153151628 | NM_001278116 | 29 | 308840 |
| ABCD1 | chrX | 152990322 | 153010216 | NM_000033 | 10 | 300371 |
| DYSF | chr2 | 71693831 | 71913893 | NM_001130983 | 56 | 603009 |
| GBA2 | chr9 | 35736862 | 35749225 | NM_020944 | 17 | 609471 |
| TRAPPC9 | chr8 | 140742585 | 141468678 | NM_031466 | 23 | 611966 |
| CYP2U1 | chr4 | 108852716 | 108874613 | NM_183075 | 5 | 610670 |
| PANK2 | chr20 | 3869741 | 3904502 | NM_153638 | 7 | 606157 |
| ARL13B | chr3 | 93698982 | 93774522 | NM_182896 | 11 | 608922 |
| KIF7 | chr15 | 90171200 | 90198682 | NM_198525 | 19 | 611254 |
| ERLIN2 | chr8 | 37594096 | 37615319 | NM_007175 | 12 | 611605 |
| PSAP | chr10 | 73576054 | 73611082 | NM_001042465 | 15 | 176801 |
| VAPB | chr20 | 56964174 | 57026156 | NM_004738 | 6 | 605704 |
| FKTN | chr9 | 108320410 | 108403399 | NM_001198963 | 12 | 607440 |
| PLP1 | chrX | 103031753 | 103047547 | NM_000533 | 7 | 300401 |
| GDAP1 | chr8 | 75262617 | 75279335 | NM_018972 | 6 | 606598 |
| ASPM | chr1 | 197053256 | 197115824 | NM_018136 | 28 | 605481 |
| LAMA2 | chr6 | 129204285 | 129837710 | NM_000426 | 65 | 156225 |
| MECP2 | chrX | 153295685 | 153363188 | NM_001110792 | 3 | 300005 |
| CDK5RAP2 | chr9 | 123151146 | 123342448 | NR_073556 | 39 | 608201 |
| WDR81 | chr17 | 1628124 | 1641893 | NM_001163809 | 10 | 614218 |
| ABAT | chr16 | 8768443 | 8878432 | NM_020686 | 16 | 137150 |
| NDE1 | chr16 | 15737124 | 15820210 | NM_017668 | 9 | 609449 |
| WDR45B | chr17 | 80572438 | 80606429 | NM_019613 | 10 | 609226 |
| HSD17B4 | chr5 | 118788201 | 118878030 | NM_001199291 | 25 | 601860 |
| HEXA | chr15 | 72635777 | 72668520 | NM_000520 | 14 | 606869 |
| SPG11 | chr15 | 44854893 | 44955876 | NM_025137 | 40 | 610844 |
| PDGFRB | chr5 | 149493401 | 149535422 | NM_002609 | 23 | 173410 |
| HUWE1 | chrX | 53559056 | 53713697 | NM_031407 | 84 | 300697 |
| SLC25A19 | chr17 | 73269060 | 73285530 | NM_001126122 | 7 | 606521 |
| ARHGEF6 | chrX | 135747711 | 135863503 | NM_004840 | 22 | 300267 |
| ADRA2B | chr2 | 96778622 | 96781888 | NM_000682 | 1 | 104260 |
| RELN | chr7 | 103112230 | 103629963 | NM_005045 | 65 | 600514 |
| CENPJ | chr13 | 25456411 | 25497027 | NR_047594 | 18 | 609279 |
| ARL14EP | chr11 | 30344598 | 30359774 | NM_152316 | 4 | 612295 |
| PHGDH | chr1 | 120254418 | 120286849 | NM_006623 | 12 | 606879 |
| ARID1B | chr6 | 157099063 | 157531913 | NM_020732 | 20 | 614556 |
| WNK1 | chr12 | 862088 | 1020618 | NM_018979 | 28 | 605232 |
| SEPN1 | chr1 | 26126666 | 26144713 | NM_020451 | 13 | 606210 |
| RNASEH2C | chr11 | 65485143 | 65488409 | NM_032193 | 4 | 610330 |
| RNASEH2B | chr13 | 51483813 | 51530901 | NM_024570 | 11 | 610326 |
| CYP27A1 | chr2 | 219646471 | 219680016 | NM_000784 | 9 | 606530 |
| ATN1 | chr12 | 7033625 | 7051484 | NM_001007026 | 10 | 607462 |
| AHI1 | chr6 | 135708921 | 135818903 | NM_001134832 | 23 | 608894 |
| STXBP1 | chr9 | 130374485 | 130454995 | NM_001032221 | 19 | 602926 |
| CDKL5 | chrX | 18460343 | 18671749 | NM_001037343 | 22 | 300203 |
| MED23 | chr6 | 131907877 | 131949379 | NM_001270522 | 30 | 605042 |
| ISPD | chr7 | 16127151 | 16460947 | NM_001101426 | 10 | 614631 |
| CEP57 | chr11 | 95523624 | 95565857 | NM_001243776 | 12 | 607951 |
| AGRN | chr1 | 955502 | 991499 | NM_198576 | 36 | 103320 |
| FKRP | chr19 | 47249302 | 47261832 | NM_001039885 | 4 | 606596 |
| ADCK3 | chr1 | 227127937 | 227175246 | NM_020247 | 15 | 606980 |
| SCN2A | chr2 | 166095911 | 166248820 | NM_001040142 | 27 | 182390 |
| MFSD8 | chr4 | 128838959 | 128887139 | NM_152778 | 13 | 611124 |
| TYMP | chr22 | 50964180 | 50968514 | NM_001257989 | 10 | 131222 |
| FLVCR2 | chr14 | 76044939 | 76114512 | NM_017791 | 10 | 610865 |
| SPG20 | chr13 | 36875774 | 36944317 | NM_001142294 | 9 | 607111 |
| CACNA1G | chr17 | 48638428 | 48704832 | NM_018896 | 38 | 604065 |
| PLA2G6 | chr22 | 38507501 | 38577761 | NM_003560 | 17 | 603604 |
| CLN6 | chr15 | 68499329 | 68522080 | NM_017882 | 7 | 606725 |
| WDR62 | chr19 | 36545782 | 36596012 | NM_173636 | 32 | 613583 |
| PEX26 | chr22 | 18560759 | 18573797 | NM_001127649 | 5 | 608666 |
| KIF1A | chr2 | 241653180 | 241759725 | NM_001244008 | 49 | 601255 |
| PNPO | chr17 | 46018888 | 46026674 | NM_018129 | 7 | 603287 |
| LARGE | chr22 | 33669061 | 34316416 | NM_133642 | 15 | 603590 |
| YARS | chr1 | 33240839 | 33283633 | NM_003680 | 13 | 603623 |
| KIAA0196 | chr8 | 126036502 | 126104061 | NM_014846 | 29 | 610657 |
| CCDC88C | chr14 | 91737666 | 91884188 | NM_001080414 | 30 | 611204 |
| OPTN | chr10 | 13142081 | 13180276 | NM_001008213 | 16 | 602432 |
| OCLN | chr5 | 68788589 | 68853931 | NM_001205254 | 9 | 602876 |
| ATRX | chrX | 76760355 | 77041719 | NM_000489 | 35 | 300032 |
| ATL1 | chr14 | 50999799 | 51099784 | NM_001127713 | 14 | 606439 |
| GNE | chr9 | 36214438 | 36277053 | NM_001128227 | 12 | 603824 |
| PEX12 | chr17 | 33901813 | 33905656 | NM_000286 | 3 | 601758 |
| SPTBN2 | chr11 | 66452719 | 66488870 | NM_006946 | 37 | 604985 |
| PEX16 | chr11 | 45931219 | 45939674 | NM_057174 | 11 | 603360 |
| COL6A1 | chr21 | 47401662 | 47424963 | NM_001848 | 35 | 120220 |
| COL6A3 | chr2 | 238232654 | 238322850 | NM_004369 | 44 | 120250 |
| COL6A2 | chr21 | 47518032 | 47552763 | NM_001849 | 28 | 120240 |
| HEPACAM | chr11 | 124789145 | 124806308 | NM_152722 | 7 | 611642 |
| LRPPRC | chr2 | 44113362 | 44223144 | NM_133259 | 38 | 607544 |
| RYR1 | chr19 | 38924339 | 39078204 | NM_000540 | 106 | 180901 |
| NTRK1 | chr1 | 156830670 | 156851642 | NM_002529 | 17 | 191315 |
| CAPN3 | chr15 | 42651697 | 42704515 | NM_000070 | 24 | 114240 |
| SOD1 | chr21 | 33031934 | 33041243 | NM_000454 | 5 | 147450 |
| COG6 | chr13 | 40229763 | 40326765 | NR_026745 | 20 | 606977 |
| ATP2B3 | chrX | 152801579 | 152848387 | NM_021949 | 21 | 300014 |
| DPYD | chr1 | 97543299 | 98386615 | NM_000110 | 23 | 612779 |
| TUBA1A | chr12 | 49578577 | 49583107 | NM_006009 | 4 | 602529 |
| TCTN1 | chr12 | 111051911 | 111086935 | NM_001173975 | 15 | 609863 |
| CPA6 | chr8 | 68334404 | 68658620 | NM_020361 | 11 | 609562 |
| ABHD12 | chr20 | 25275378 | 25371618 | NM_015600 | 13 | 613599 |
| NPC2 | chr14 | 74946642 | 74960084 | NM_006432 | 5 | 601015 |
| MPDZ | chr9 | 13105702 | 13279563 | NM_001261406 | 46 | 603785 |
| SYNGAP1 | chr6 | 4868092 | 4901710 | NM_006772 | 19 | 603384 |
| PEX5 | chr12 | 7341758 | 7371169 | NM_001131026 | 18 | 600414 |
| PEX6 | chr6 | 42931610 | 42946981 | NM_000287 | 17 | 601498 |
| POMT1 | chr9 | 134378288 | 134399193 | NM_001077365 | 20 | 607423 |
| POMT2 | chr14 | 77741298 | 77787225 | NM_013382 | 21 | 607439 |
| MCPH1 | chr8 | 6264112 | 6501140 | NM_024596 | 14 | 607117 |
| CASC5 | chr15 | 40886446 | 40954881 | NM_170589 | 27 | 609173 |
| SGCB | chr4 | 152886860 | 52904485 | NM_000232 | 6 | 600900 |
| SGCA | chr17 | 48243365 | 48253293 | NM_000023 | 10 | 600119 |
| POMGNT2 | chr3 | 43120724 | 43147568 | NM_032806 | 2 | 614828 |
| TRMT1 | chr19 | 13215713 | 13227563 | NM_001136035 | 17 | 611669 |
| ARFGEF2 | chr20 | 47538274 | 47653230 | NM_006420 | 39 | 605371 |
| SYNE2 | chr14 | 64319682 | 64693167 | NM_182914 | 116 | 608442 |
| ADK | chr10 | 75910942 | 76469061 | NM_006721 | 11 | 102750 |
| ZNF526 | chr19 | 42724491 | 42732353 | NM_133444 | 3 | 614387 |
| FOXG1 | chr14 | 29236277 | 29239483 | NM_005249 | 1 | 164874 |
| ALS2 | chr2 | 202564985 | 202645895 | NM_020919 | 34 | 606352 |
| C5orf42 | chr5 | 37106329 | 37249530 | NM_023073 | 52 | 614571 |
| TMEM237 | chr2 | 202484906 | 202508252 | NM_001044385 | 12 | 614423 |
| C12orf57 | chr12 | 7053202 | 7055165 | NM_138425 | 3 | 615140 |

For detection of pelvic inflammatory disease (PID) (or the proclivity therefor), the at least one genetic marker is selected from the group consisting of IL7R, JAK3, CD40LG, AK2, DCLRE1C, CD40, AICDA, MLPH, NHEJ1, RAB27A, RAG2, RAG1, BTK, ATM, LYST, CYBB, AIRE, DOCK8, SLC17A5, STAT3, WAS, CD247, DNMT3B, FLG, NCF2, ADA, RFXANK, PTPRC, COLEC11 and combinations thereof. The details of the pelvic inflammatory disease panel are given below in Table 10.

TABLE 10
Pelvic Inflammatory Disease (PID) Panel

| | | | | | Number | |
|---|---|---|---|---|---|---|
| | | | | | of | |
| Gene | Chr | Start | End | Transcript | Exons | MIM |
| IL7R | chr5 | 35856976 | 35879705 | NM_002185 | 8 | 146661 |
| JAK3 | chr19 | 17935592 | 17958841 | NM_000215 | 24 | 600173 |
| CD40LG | chrX | 135730335 | 135742549 | NM_000074 | 5 | 300386 |
| AK2 | chr1 | 33473540 | 33502512 | NR_037591 | 8 | 103020 |
| DCLRE1C | chr10 | 14948870 | 14996094 | NM_001033858 | 16 | 605988 |
| CD40 | chr20 | 44746905 | 44758384 | NM_001250 | 9 | 109535 |
| AICDA | chr12 | 8754761 | 8765442 | NM_020661 | 5 | 605257 |
| MLPH | chr2 | 238395877 | 238463961 | NM_024101 | 16 | 606526 |
| NHEJ1 | chr2 | 219940045 | 220025587 | NM_024782 | 8 | 611290 |
| RAB27A | chr15 | 55495163 | 55582013 | NM_183235 | 7 | 603868 |
| RAG2 | chr11 | 36613492 | 36619829 | NM_000536 | 2 | 179616 |
| RAG1 | chr11 | 36589562 | 36601310 | NM_000448 | 2 | 179615 |
| BTK | chrX | 100604434 | 100641212 | NM_000061 | 19 | 300300 |
| ATM | chr11 | 108093558 | 108239826 | NM_000051 | 63 | 607585 |
| LYST | chr1 | 235824330 | 236030227 | NM_000081 | 53 | 606897 |
| CYBB | chrX | 37639269 | 37672714 | NM_000397 | 13 | 300481 |
| AIRE | chr21 | 45705720 | 45718102 | NM_000383 | 14 | 607358 |
| DOCK8 | chr9 | 214864 | 465259 | NM_203447 | 48 | 611432 |
| SLC17A5 | chr6 | 74303101 | 74363737 | NM_012434 | 11 | 604322 |
| STAT3 | chr17 | 40465342 | 40540405 | NM_213662 | 24 | 102582 |
| WAS | chrX | 48542185 | 48549817 | NM_000377 | 12 | 300392 |
| CD247 | chr1 | 167399876 | 167487847 | NM_198053 | 8 | 186780 |
| DNMT3B | chr20 | 31350190 | 31397162 | NM_006892 | 23 | 602900 |
| FLG | chr1 | 152274650 | 152297679 | NM_002016 | 3 | 135940 |
| NCF2 | chr1 | 183524696 | 183560056 | NM_001127651 | 16 | 608515 |
| ADA | chr20 | 43248162 | 43280376 | NM_000022 | 12 | 608958 |
| RFXANK | chr19 | 19303007 | 19312678 | NM_003721 | 10 | 603200 |
| PTPRC | chr1 | 198608097 | 198726605 | NM_002838 | 33 | 151460 |

For detection of pulmonary conditions (or the proclivity therefor), the at least one genetic marker is selected from the group consisting of SFTPB, CFTR and combinations thereof. The details of the pulmonary panel are given below in Table 11.

TABLE 11
Pulmonary Panel

| Gene | Chr | Start | End | Transcript | Number of Exons | MIM |
|---|---|---|---|---|---|---|
| SFTPB | chr2 | 85884439 | 85895864 | NM_000542 | 12 | 178640 |
| CFTR | chr7 | 117120016 | 117308718 | NM_000492 | 27 | 602421 |

For detection of renal conditions (or the proclivity therefor), the at least one genetic marker is selected from the group consisting of IQCB1, COL4A6, NPHP3, SLC4A4, DDX39A, SMARCAL1, PKHD1, LAMB2, NEK8, NPHP4, FRAS1, XDH, MKS1, FAN1, TCTN2, NPHS1, CC2D2A, TMEM231, UPK3A, CEP290, NPHP4, COL4A4, TMEM67, C5orf42, TMEM237 and combinations thereof. The details of the renal panel are given below in Table 12.

TABLE 12
Renal Panel

| | | | | | Number | |
|---|---|---|---|---|---|---|
| | | | | | of | |
| Gene | Chr | Start | End | Transcript | Exons | MIM |
| IQCB1 | chr3 | 121488609 | 121553926 | NM_001023570 | 15 | 609237 |
| COL4A6 | chrX | 107398836 | 107682704 | NM_001847 | 45 | 303631 |
| NPHP3 | chr3 | 132399452 | 132441303 | NM_153240 | 27 | 608002 |
| SLC4A4 | chr4 | 72053002 | 72437804 | NM_001098484 | 26 | 603345 |
| DDX39A | chr19 | 14519609 | 14529906 | NR_038336 | 12 | NA |
| SMARCAL1 | chr2 | 217277472 | 217347774 | NM_001127207 | 18 | 606622 |
| PKHD1 | chr6 | 51585646 | 51952423 | NM_170724 | 61 | 606702 |
| LAMB2 | chr3 | 49158546 | 49170599 | NM_002292 | 32 | 150325 |
| NEK8 | chr17 | 27055831 | 27069784 | NM_178170 | 15 | 609799 |
| NPHP4 | chr1 | 5922869 | 6052533 | NM_015102 | 30 | 607215 |
| FRAS1 | chr4 | 78978723 | 79465423 | NM_025074 | 74 | 607830 |
| XDH | chr2 | 31557187 | 31637611 | NM_000379 | 36 | 607633 |
| MKS1 | chr17 | 56282796 | 56296966 | NM_001165927 | 18 | 609883 |
| FAN1 | chr15 | 31196075 | 31203991 | NM_001146096 | 4 | 613534 |
| TCTN2 | chr12 | 124155659 | 124192950 | NM_001143850 | 18 | 613846 |
| NPHS1 | chr19 | 36316273 | 36342895 | NM_004646 | 29 | 602716 |
| CC2D2A | chr4 | 15471488 | 15603180 | NM_001080522 | 38 | 612013 |
| TMEM231 | chr16 | 75572014 | 75590184 | NM_001077416 | 6 | 614949 |
| UPK3A | chr22 | 45680867 | 45691755 | NM_006953 | 6 | 611559 |
| CEP290 | chr12 | 88442789 | 88535993 | NM_025114 | 54 | 610142 |
| NPHP4 | chr1 | 5922869 | 6052533 | NM_015102 | 30 | 607215 |

For detection of vision disorders (or the proclivity therefor), the at least one genetic marker is selected from the group consisting of ALMS1, CRB1, CHST6, CRYBA1, PRSS56, GUCY2D, SNRNP200, PDE6C, CNGA3, C8orf37, ABCA4, BBS10, CERKL, GPR125, NHS, LTBP2, GCNT2, RLBP1, MIP, RP1L1, CHM, EYS, TULP1, IGFBP7, CYP1B1, LRAT, MERTK, CNNM4, RP1, RP2, LCA5, MFRP, CNGB1, CACNA1F, KCNV2, CRX, PROM1, TRPM1, PAX6, IMPG2, CDHR1, GPR179, CRYGC, CRYGD, NMNAT1, GALT, ARL6, LRP5, WDR19, SLC4A11, GDF3, SLC16A12, RGS9, RDH12, ADAMS, AIPL1, FAM161A, RPGRIP1, RAB3GAP2, RAB3GAP1, EFEMP1, BEST1, RPE65, EPHA2, FZD4, PRPH2, CRYAA, KCNJ13, NR2E3, BBS9, BBS1, BBS2, BBS5, BBS4, BBS7, SPATA7, CHD7, USH2A, MYO7A, C12orf57, CEP290, NPHP4 and combinations thereof. The details of the vision panel are given below in Table 13.

TABLE 13
Vision Panel

| | | | | | Number | |
|---|---|---|---|---|---|---|
| | | | | | of | |
| Gene | Chr | Start | End | Transcript | Exons | MIM |
| ALMS1 | chr2 | 73612885 | 73837046 | NM_015120 | 23 | 606844 |
| CRB1 | chr1 | 197170591 | 197447585 | NM_001257965 | 15 | 604210 |
| CHST6 | chr16 | 75505950 | 75529282 | NM_021615 | 3 | 605294 |
| CRYBA1 | chr17 | 27573874 | 27581502 | NM_005208 | 6 | 123610 |
| PRSS56 | chr2 | 233385172 | 233390425 | NM_001195129 | 13 | 613858 |
| GUCY2D | chr17 | 7905987 | 7923658 | NM_000180 | 20 | 600179 |
| SNRNP200 | chr2 | 96940073 | 96971307 | NM_014014 | 45 | 601664 |
| PDE6C | chr10 | 95372344 | 95425429 | NM_006204 | 22 | 600827 |
| CNGA3 | chr2 | 98962617 | 99015064 | NM_001298 | 8 | 600053 |
| C8orf37 | chr8 | 96257140 | 96281462 | NM_177965 | 6 | 614477 |
| ABCA4 | chr1 | 94458393 | 94586705 | NM_000350 | 50 | 601691 |
| BBS10 | chr12 | 76738265 | 76742222 | NM_024685 | 2 | 610148 |
| CERKL | chr2 | 182401400 | 182521834 | NM_001030311 | 14 | 608381 |
| GPR125 | chr4 | 22388996 | 22517677 | NM_145290 | 19 | 612303 |
| NHS | chrX | 17653412 | 17754113 | NM_001136024 | 9 | 300457 |
| LTBP2 | chr14 | 74964885 | 75079034 | NM_000428 | 36 | 602091 |
| GCNT2 | chr6 | 10585992 | 10629601 | NM_145655 | 3 | 600429 |
| RLBP1 | chr15 | 89753097 | 89764922 | NM_000326 | 9 | 180090 |
| MIP | chr12 | 56843285 | 56848435 | NM_012064 | 4 | 154050 |
| RP1L1 | chr8 | 10463859 | 10512617 | NM_178857 | 4 | 608581 |
| CHM | chrX | 85116184 | 85302566 | NM_000390 | 15 | 300390 |
| EYS | chr6 | 64429875 | 66417118 | NM_001142800 | 43 | 612424 |
| TULP1 | chr6 | 35465650 | 35480647 | NM_003322 | 15 | 602280 |
| IGFBP7 | chr4 | 57897236 | 57976551 | NM_001553 | 5 | 602867 |
| CYP1B1 | chr2 | 38294745 | 38303323 | NM_000104 | 3 | 601771 |
| LRAT | chr4 | 155665162 | 155674270 | NM_004744 | 3 | 604863 |
| MERTK | chr2 | 112656190 | 112786945 | NM_006343 | 19 | 604705 |
| CNNM4 | chr2 | 97426638 | 97477628 | NM_020184 | 7 | 607805 |
| RP1 | chr8 | 55528626 | 55543394 | NM_006269 | 4 | 603937 |
| RP2 | chrX | 46696346 | 46741791 | NM_006915 | 5 | 300757 |
| LCA5 | chr6 | 80194707 | 80247147 | NM_001122769 | 8 | 611408 |
| MFRP | chr11 | 119209643 | 119217383 | NM_015645 | 15 | 606227 |
| CNGB1 | chr16 | 57916243 | 58005020 | NM_001297 | 33 | 600724 |
| CACNA1F | chrX | 49061522 | 49089771 | NM_001256790 | 48 | 300110 |
| KCNV2 | chr9 | 2717525 | 2730037 | NM_133497 | 2 | 607604 |
| CRX | chr19 | 48325098 | 48346586 | NM_000554 | 4 | 602225 |
| PROM1 | chr4 | 15969848 | 16077741 | NM_006017 | 27 | 604365 |
| TRPM1 | chr15 | 31293263 | 31393929 | NM_001252024 | 28 | 603576 |
| PAX6 | chr11 | 31806339 | 31833731 | NM_001258463 | 14 | 607108 |
| IMPG2 | chr3 | 100941389 | 101039419 | NM_016247 | 19 | 607056 |
| CDHR1 | chr10 | 85954390 | 85979376 | NM_001171971 | 17 | 609502 |
| GPR179 | chr17 | 36481492 | 36499693 | NM_001004334 | 11 | 614515 |
| CRYGC | chr2 | 208992860 | 208994554 | NM_020989 | 3 | 123680 |
| CRYGD | chr2 | 208986330 | 208989313 | NM_006891 | 3 | 123690 |
| NMNAT1 | chr1 | 10003485 | 10045556 | NM_022787 | 5 | 608700 |
| GALT | chr9 | 34646585 | 34650595 | NM_000155 | 11 | 606999 |
| ARL6 | chr3 | 97483364 | 97520086 | NR_103511 | 10 | 608845 |
| LRP5 | chr11 | 68080107 | 68216743 | NM_002335 | 23 | 603506 |
| WDR19 | chr4 | 39184023 | 39287430 | NM_025132 | 37 | 608151 |
| SLC4A11 | chr20 | 3208062 | 3219887 | NM_001174089 | 20 | 610206 |
| GDF3 | chr12 | 7842380 | 7848360 | NM_020634 | 2 | 606522 |
| SLC16A12 | chr10 | 91190050 | 91295313 | NM_213606 | 8 | 611910 |
| RGS9 | chr17 | 63133455 | 63223821 | NM_001081955 | 19 | 604067 |
| RDH12 | chr14 | 68168602 | 68201168 | NM_152443 | 9 | 608830 |
| ADAM9 | chr8 | 38854504 | 38962779 | NM_003816 | 22 | 602713 |
| AIPL1 | chr17 | 6327058 | 6338519 | NM_014336 | 6 | 604392 |
| FAM161A | chr2 | 62051982 | 62081278 | NM_001201543 | 7 | 613596 |
| RPGRIP1 | chr14 | 21756135 | 21819460 | NM_020366 | 24 | 605446 |
| RAB3GAP2 | chr1 | 220321609 | 220445843 | NM_012414 | 35 | 609275 |
| RAB3GAP1 | chr2 | 135809834 | 135928279 | NM_001172435 | 25 | 602536 |
| EFEMP1 | chr2 | 56093096 | 56151298 | NM_001039349 | 11 | 601548 |
| BEST1 | chr11 | 61717355 | 61731935 | NM_004183 | 11 | 607854 |
| RPE65 | chr1 | 68894506 | 68915642 | NM_000329 | 14 | 180069 |
| EPHA2 | chr1 | 16450831 | 16482582 | NM_004431 | 17 | 176946 |
| FZD4 | chr11 | 86656716 | 86666440 | NM_012193 | 2 | 604579 |
| PRPH2 | chr6 | 42664332 | 42690358 | NM_000322 | 3 | 179605 |
| CRYAA | chr21 | 44589140 | 44592913 | NM_000394 | 3 | 123580 |
| KCNJ13 | chr2 | 233631174 | 233641278 | NM_002242 | 3 | 603208 |
| NR2E3 | chr15 | 72102893 | 72110597 | NM_014249 | 9 | 604485 |
| BBS9 | chr7 | 33169151 | 33645680 | NM_198428 | 23 | 607968 |
| BBS1 | chr11 | 66278077 | 66301098 | NM_024649 | 17 | 209901 |
| BBS2 | chr16 | 56518258 | 56554008 | NM_031885 | 17 | 606151 |
| BBS5 | chr2 | 170336005 | 170363165 | NM_152384 | 12 | 603650 |
| BBS4 | chr15 | 72978519 | 73030817 | NR_045565 | 17 | 600374 |
| BBS7 | chr4 | 122748881 | 122791652 | NM_018190 | 18 | 607590 |
| SPATA7 | chr14 | 88851987 | 88904804 | NM_018418 | 12 | 609868 |
| CHD7 | chr8 | 61591323 | 61780586 | NM_017780 | 38 | 608892 |

642 samples with known mutations were used to calculate the analytical sensitivity of the Mendeliome assay. Overall analytical sensitivity was 79% (507/642). One hundred and thirty-five known mutations were missed by the Mendeliome assay, 46% (62/135) of which were due to a design flaw; i.e., the disease gene was not included in the panel appropriate for the disease presentation. If these 62 cases were to be excluded, the overall analytical sensitivity would increase to 87% (507/580). Based on these positive controls (580), sensitivity for single nucleotide variants was found to be 93% (398/428). However, sensitivity for indels was lower at 72% (109/152). As expected for semiconductor-based Ion Torrent sequencing, the bias against indels was not uniform but was largely sequence context-dependent, especially around homopolymer region.

In addition to these positive controls, single nucleotide polymorphism (SNP) genotyping arrays were used (Affymetrix Axiom GT1 chip with ˜580,000 SNPs) coming from 21 patients as a second method of testing the analytical sensitivity. The variants detected by SNP arrays were compared to those detected by the next generation sequencing (NGS) technology for each sample. From a total of 3,319 SNPs lying within the target regions of the panels, the resulting SNP sensitivity was about 95%. Interestingly, 30 extra SNPs were identified that were called by the assay but were not called with high confidence on the chip. For analytical specificity, a predetermined quality score of 100 was used (this takes into account strand-bias, homopolymer errors, etc.). Analytical specificity was based on the Sanger validation of 1,078 variants called by the assay. Sanger sequencing confirmed 93% (819/881) of SNVs and 78% (154/197) of indels that met or were higher than that quality score.

A total of 2,357 patients representing a very wide range of suspected genetic diseases were tested by the Mendeliome assay (see Table 14 below for the number of patients tested on each panel). Only one panel was chosen per patient based on the most prominent primary clinical feature. The overall clinical sensitivity (i.e., detection of a likely causal variant that is subsequently confirmed by Sanger sequencing) was 43%. Table 14 also summarizes the clinical sensitivity per panel as well as per clinical feature within each panel. As expected, specialties with the highest referral rate were neurology, dysmorphology, pediatric ophthalmology and immunology because of the nonspecificity of the clinical presentation, extreme and genetic heterogeneity, and because a genetic cause is highly suspected for a large fraction of their patient population. In fact, a relatively high yield for the respective panels of 40%, 38%, 52%, and 37% were noted (see Table 14). Specificity of the presentation appeared to bear appreciably on the clinical sensitivity of the assay. For example, with an objective evidence of skeletal dysplasia the sensitivity of the dymorphology/dysplasia panel was 45% as compared to 32% when any degree of dysmorphism was used as the entry point. Similarly, the finding of a specific pattern of neurological abnormality (e.g., muscular dystrophy and neurodegenerative disorders) was associated with a much higher sensitivity as compared with non-syndromic developmental delay/intellectual disability of any degree (56% and 42% vs 11%). Also consistent with this is the finding that retinal dystrophies (almost always Mendelian in etiology) were more likely to have positive hits than the overall performance of the fision panel (65% vs 52%).

TABLE 14
Clinical Sensitivities Per Panel

| | Total | Overall | | |
|---|---|---|---|---|
| | Patients | Clinical | | |
| Gene Panel Type | Run | Sensitivity | Selected Subgroup Clinical Sensitivity | |
| Cardiovascular | 243 | 28% | Cardiomyopathy | 32% |
| | | | Congenital heart disease | 10% |
| | | | Arrhythmias | 31% |
| | | | Aneurysms | 29% |
| Deafness | 147 | 54% | Hearing Loss | — |
| Dermatology | 68 | 62% | Nonspecific Dermatological | — |
| | | | Features | |
| Dysmorphology- | 354 | 38% | Skeletal dysplasia | 45% |
| Dysplasia | | | Dysmorphism | 32% |
| Endocrinology | 36 | 61% | Pituitary and Thyroid Disorders | — |
| Gastroenterology | 73 | 29% | Persistent Jaundice | — |
| Hematology | 33 | 24% | Aplastic Anemia | — |
| Inborn errors of | 122 | 59% | Metabolic disorders | — |
| metabolism | | | | |
| Neurology | 524 | 40% | Syndromic DD/ID_Recognizable | 47% |
| | | | syndromes | |
| | | | Syndromic DD/ID NYD (not yet | 25% |
| | | | determined)_Unrecognizable | |
| | | | syndrome | |
| | | | Structural Brain | 34% |
| | | | (Cerebral/Cerebellar/brain stem) | |
| | | | and spinal | |
| | | | malformations/anomalies*1 | |
| | | | Non Syndromic DD/ID NYD (not | 11% |
| | | | yet determined, | |
| | | | unrecognizable)*2 | |
| | | | Neurodegenerative disorders | 42% |
| | | | Coordination*3/Movement | 69% |
| | | | disorders | |
| | | | Peripheral neuropathy | 33% |
| | | | Myopathies/Joint abnormalities*4 | 56% |
| PID | 196 | 37% | Primary immunodeficiency | — |
| | | | disorders | |
| Pulmonology | 36 | 36% | Chronic lung infection suspected | — |
| | | | cystic fibrosis | |
| Renal | 107 | 57% | Glomerular/Tubular Disorders | 41% |
| | | | Cystic Kidney Disease | 63% |
| | | | Kidney Malformation | 69% |
| Vision | 418 | 52% | Retinal dystrophy (syndromic, | 65% |
| | | | non-syndromic, RP, CRD, | |
| | | | macular dystrophy, FEVR, GFS) | |
| | | | Cataract (syndromic and non- | 34% |
| | | | syndromic) | |
| | | | Aniridia | 33% |
| | | | Microphthalmia/anophthalmia | 30% |
| | | | (with and without coloboma) | |
| | | | Corneal dystrophy (CHED and all | 40% |
| | | | other subtypes) | |
| | | | Others | 23% |

*1Primary microcephaly cases are included in this group,
*2Non syndromic cases of Autism/mental disorder and epilepsy are included under this group,
*3Ataxia cases secondary to cerebellar hypoplasia are included under the structural brain abnormalities group,
*4Cases with Arthrogryposis Multiplex syndromes are included under myopathies group.
PID: Primary immunodeficiency,
DD: developmental delay,
ID: intellectual disability,
RP: retinitis pigmentosa,
CRD: cone-rod dystrophy,
FEVR: familial exudative vitreoretinopathy,
GFS: Goldmann-Favre syndrome,
CHED: corneal hereditary endothelial dystrophy

The clinical sensitivity of the Mendeliome assay (43%) is comparable to the ˜25% reported by several large clinical whole exome sequencing (WES) studies. The Mendeliome assay is inherently limited to established disease genes, so it will miss cases caused by large structural variants and mutations in novel genes, although the design is flexible and allows for the addition of newly published disease genes as frequently as needed, e.g. every six months. 213 cases were randomly selected that were negative by the Mendeliome assay, and these were processed using molecular karyotyping. Thirty-five of these were found to have likely pathogenic de novo copy-number variations (CNVs). If these 35 cases are excluded, the clinical sensitivity of the present method would increase slightly to 44%. The remaining 178 were processed using WES, and only 11% (20/178) were found by WES to have a mutation in a known gene that was missed by the Mendeliome assay. Out of these 20 missed cases, the majority (n=14, 70%) were due to a design flaw (i.e., the disease gene was not included in the panel appropriate for the disease presentation) and this can easily be fixed by a spike-in approach.

The remaining six cases represent a limitation of the analytical sensitivity of the next-generation sequencing platform used in this study. On the other hand, it should be noted that two patients were included who had had negative diagnostic WES results prior to their enrollment in the Mendeliome assay, and were found to have likely causal mutations by the latter. These cases were missed at the interpretation phase of WES analysis and were solved by the Mendeliome assay, likely because of the smaller number of variants. The much smaller number of variants to be queried by the Mendeliome assay vs. WES also meant a much more rapid clinical interpretation (average 20 min per panel vs. 2-3 hours per WES). This has markedly reduced the cost of interpretation on top of an already appreciable reduction in running cost (24 panel samples were run per chip vs. one WES per chip). The cost is estimated to be $150 per sample with a range of $75-$150 per sample depending on the panel selected. The cost difference is even more dramatic for de novo mutations (n=31) that we identified in this study, because they are typically identifiable by WES only when a trio design is followed. These de novo mutations were identifiable as likely disease-causing heterozygous mutations in relevant Mendelian genes, and their de novo status was confirmed by Sanger sequencing of a single amplicon in both parents. Also relevant to cost reduction is that five couples who lost children with a likely recessive disease were used, but there was no access to DNA from the deceased children. By running the appropriate panel on both parents the method was able to identify the likely causal mutation at a much lower cost than the duo WES design that would have been required to reach the same conclusion.

WES is frequently requested after one or more genes deemed relevant to the patient's clinical presentation had been excluded by Sanger sequencing in hopes of identifying a novel genetic cause. However, many WES studies have highlighted the frequent encounter of disease-causing mutations in known genes that would not have been considered good candidates owing to the marked discrepancy between their published phenotype and the clinical presentation of the patient especially for neurological and dysmorphic disorders, which are often very heterogeneous clinically. It has been shown that even in familial cases that are carefully enriched for novel gene discovery by excluding all relevant candidate genes by autozygome analysis, 11% of WES will reveal mutations in known genes missed by the enrichment step because the presentation was very atypical. In fact, in many patients with disease-causing mutations identified by the Mendeliome assay, the presentation was sufficiently different from the published phenotype of the respective gene that WES would have been pursued to establish the diagnosis (see Table 15 below). Some of the most dramatic examples are a de novo EP300 mutation causing microcephalic primordial dwarfism, a homozygous ZNF526 mutation causing a novel Noonan-like phenotype, a homozygous IFT122 mutation causing severe ocular anomalies and unusual appendicular skeletal abnormalities, and a de novo KMT2A mutation causing genital abnormalities in an affected female including absent uterus and vagina with remarkable clitoromegaly (see Table 15).

On the other hand, mutations in genes were identified which are typically associated with multisystem disorders in patients with a very limited phenotype, e.g., NPHP4 mutation in a patient with isolated retinal dystrophy instead of Senior-Loken syndrome, and RAB3GAP1 causing isolated cataract instead of Warburg Micro syndrome (Table 15). Finally, it should be noted that the highly surprising finding of a homozygous nonsense mutation in TCOF1 causing severe Treacher-Collins syndrome while the carrier parents are completely normal clinically. Interestingly, this mutation had been missed by direct Sanger sequencing of TCOF1, most likely because the expectation was a heterozygous peak on the sequence chromatogram given the dominant nature of the disease. This is the first instance of a recessive inheritance of TCOF1.

TABLE 15
Atypical Phenotypes

| | | | | | | Observed Phenotype compared to | |
|---|---|---|---|---|---|---|---|
| | | | | | | published phenotype(s) | |
| | | | | | | Typical/ | |
| | | | | | Published | Previously | |
| Gene | | Mutation | | | Phenotype(s) related | reported | Atypical |
| Name | Type | Status | Origin | Type | to the case | feature(s) | feature(s) |
| FGFR2 | Missense | HTZ | De | DD | Craniosynostosis | Craniofacial | Upper eyelid |
| | | | Novo | | syndromes | anomalies | coloboma |
| | | | | | | Sacrococcygeal | |
| | | | | | | tail | |
| | | | | | | Syndactyly | |
| | | | | | | Neonatal | |
| | | | | | | teeth (with | |
| | | | | | | Beare- | |
| | | | | | | Stevenson | |
| | | | | | | Cutis Gyrata | |
| | | | | | | Syndrome) | |
| | | | | | | Choanalatresia | |
| | | | | | | Thinning of | |
| | | | | | | the genu of | |
| | | | | | | corpus | |
| | | | | | | callosum (with | |
| | | | | | | Pfeiffer | |
| | | | | | | Syndrome) | |
| HRAS | Missense | HTZ | De | DD | Costello syndrome | Dysmorphic | Corneal |
| | | | Novo | | | facies | haziness |
| | | | | | | Multiple joint | Tracheomalacia |
| | | | | | | dislocations | and |
| | | | | | | | bronchomalacia |
| COL2A1 | Missense | HTZ | De | DD | SpondyloMetaepiphyseal | Disproportionate | Valvular |
| | | | Novo | | dysplasia | short | disease (mild |
| | | | | | | stature | TR, MR) |
| | | | | | | Thoracic | Acanthosis |
| | | | | | | dextroscoliosis | nigricans |
| | | | | | | Metaphyseal | |
| | | | | | | dysplasia | |
| | | | | | | Inguinal | |
| | | | | | | hernia | |
| | | | | | | Myopia | |
| TCOF1 | Nonsense | HMZ | Inherited | DD | Treacher Collins | Underdevelopment | Confirmed to |
| | | | | | syndrome 1 | zygoma | be inherited as |
| | | | | | | Choanalatresia | AR |
| | | | | | | Microtia | |
| | | | | | | No external | |
| | | | | | | auditory | |
| | | | | | | meatus | |
| | | | | | | Malformed | |
| | | | | | | ossicles and | |
| | | | | | | semicircular | |
| | | | | | | canal | |
| | | | | | | Iris/optic disc | |
| | | | | | | coloboma | |
| BRAF | Missense | HTZ | De | DD | Cardiofaciocutaneous | Hypotonia | Coarse face |
| | | | Novo | | syndrome | Speech delay | similar to |
| | | | | | | | Costello |
| | | | | | | | syndrome |
| | | | | | | | No cardiac |
| | | | | | | | defects |
| | | | | | | | Acanthosis |
| | | | | | | | nigricans |
| | | | | | | | Deep palmar |
| | | | | | | | and plantar |
| | | | | | | | creases |
| EP300 | Nonsense | HTZ | De | DD | Rubinstein-Taybi | None | Atypical |
| | | | Novo | | syndrome 2 | | dysmorphic |
| | | | | | | | facies |
| | | | | | | | Microcephalic |
| | | | | | | | primordial |
| | | | | | | | dwarfism |
| NFIX | Nonsense | HTZ | Un- | DD | Sotos Syndrome type 2 | Overgrowth | Marfanoid |
| | | | known | | | GDD | Habitus |
| | | | Father | | | | Normal bone |
| | | | was not | | | | age |
| | | | tested | | | | |
| GNS | Frame- | HMZ | Inherited | DD | Mucopolysaccharidosis | Mild coarse | Advanced RP |
| | shift | | | | type IIID | face | |
| | | | | | | Mild | |
| | | | | | | hepatomegaly | |
| | | | | | | Clear cornea | |
| | | | | | | Skeletal | |
| | | | | | | manifestations | |
| COL11A2 | Missense | HMZ | Inherited | DD | Otospondylomegaepiphyseal | Epiphyseal | Hypoplastic |
| | | | | | dysplasia | dysplasia | optic nerve |
| | | | | | | CP | Mitral valve |
| | | | | | | Deafness | prolapse and |
| | | | | | | | regursitation |
| IFT122 | Splice | HMZ | Inherited | DD | Cranioectodermal | Nystagmus | Iris and optic |
| | site | | | | dysplasia 1 | Metaphyseal | nerve coloboma |
| | | | | | | dysplasia | Microphthalmia |
| | | | | | | | Duplicated |
| | | | | | | | thumb and big |
| | | | | | | | toe |
| | | | | | | | Post-axial |
| | | | | | | | polydactyly |
| | | | | | | | Very short |
| | | | | | | | tibiae compared |
| | | | | | | | to fibulae |
| ROR2 | Missense | HMZ | Parents | DD | Robinow syndrome | Vertebral | Atypical |
| | | | not | | Brachydactyly, type | anomalies (in | fibrochondroge |
| | | | tested | | B1 | Robinow | nesis-like |
| | | | | | | syndrome) | skeletal |
| | | | | | | | dysplasia |
| KMT2A | Frame- | HTZ | De | DD | Wiedemann-Steiner | Dysmorphic | Absent uterus |
| | shift | | Novo | | syndrome | facies | and vagina, |
| | | | | | | | remarkable |
| | | | | | | | clitoromegaly |
| NSD1 | Nonsense | HTZ | De | DD | Sotos Syndrome 1 | Dysmorphic | No overgrowth |
| | | | Novo | | | facies | |
| | | | | | | Thin corpus | |
| | | | | | | callosum, | |
| | | | | | | PVL and | |
| | | | | | | colpocephaly | |
| | | | | | | on MRI brain | |
| NPHP4 | Missense | HMZ | Inherited | Vision | Senior_Loken | RP | Lack of |
| | | | | | syndrome | | systemic |
| | | | | | | | involvement |
| | | | | | | | (isolate RP) |
| CNNM4 | Missense | HMZ | Parents | Vision | Jalili syndrome | LCA, retinal | Retinal |
| | | | not | | | degeneration | coloboma |
| | | | tested | | | | No dental |
| | | | | | | | anomalies |
| BBS4 | Frame- | Compound | Parents | Vision | Bardet-Biedl | RP | Lack of other |
| | shift | HTZ | not | | syndrome 4 | | features of BBS |
| | | | tested | | | | (isolated RP) |
| RAB3GAP1 | Nonsense | HMZ | Likely | Vision | Warburg micro | Congenital | Lack of |
| | | | inherited | | syndrome 1 | cataract | microcephaly |
| | | | | | | | and severe |
| | | | | | | | ocular |
| | | | | | | | anomalies |
| ATRX | Nonsense | Hemizygous | Inherited | Vision | Mental retardation- | Microcephaly | RP |
| | | | | | hypotonic facies | GDD | Optic disc |
| | | | | | syndrome, X-linked | White matter | coloboma |
| | | | | | | changes | |
| ALMS1 | Frame- | HMZ | Inherited | Vision | Alstrom syndrome | Achromatopsia | Lack of other |
| | shift | | | | | | features of |
| | | | | | | | Alstrom |
| | | | | | | | syndrome |
| | | | | | | | (isolated |
| | | | | | | | achromatopsia) |
| STXBP1 | Missense | HTZ | De | Neuro | Epileptic | Seizures | Pigmentary |
| | | | Novo | | encephalopathy, early | | retinal changes |
| | | | | | infantile, 4 | | |
| CDKL5 | Nonsense | HMZ | Inherited | Neuro | Epileptic | GDD | Macrocephaly |
| | | | | | encephalopathy, early | | and overgrowth |
| | | | | | infantile, 2 | | Facial |
| | | | | | | | dysmorphism |
| | | | | | | | similar to Sotos |
| | | | | | | | syndrome but |
| | | | | | | | normal bone |
| | | | | | | | age and |
| | | | | | | | negative NSD1 |
| | | | | | | | mutation |
| | | | | | | | No Seizures or |
| | | | | | | | regression |
| | | | | | | | No abnormal |
| | | | | | | | movements |
| GTDC2 | Missense | HMZ | Inherited | Neuro | Muscular dystrophy- | None | Isolated large |
| (POMGNT2) | | | | | dystroglycanopathy | | occipital |
| | | | | | (congenital with brain | | encephalocele |
| | | | | | and eye anomalies, | | No polydactyly |
| | | | | | type A, 8 | | Neonatal death |
| HSD17B4 | Missense | HMZ | Inherited | Neuro | D-bifunctional protein | Neonatal | Normal brain |
| | | | | | deficiency | seizures | MRI |
| | | | | | | | No skeletal |
| | | | | | | | manifestations |
| | | | | | | | or stippling |
| | | | | | | | No eye |
| | | | | | | | findings |
| | | | | | | | No |
| | | | | | | | dysmorphism |
| ATN1 | Missense | HTZ | De | Neuro | Dentatorubro- | None | Early onset |
| | | | Novo | | pallidoluysian atrophy | | static |
| | | | | | | | encephalopathy |
| | | | | | | | Novel |
| | | | | | | | molecular |
| | | | | | | | mechanism |
| | | | | | | | (point mutation) |
| KIAA0196 | Missense | HTZ | De | Neuro | Ritscher-Schinzel | Seizures | Normal brain |
| | | | Novo | | syndrome | Speech delay | MRI |
| | | | | | Spastic paraplegia 8, | and learning | No ataxia or |
| | | | | | AD | disability | spasticity |
| ADRA2B | Nonsense | HMZ | Inherited | Neuro | Non-syndromic ID | None | Microcephaly |
| | | | | | (Najmabadi et al, | | GDD |
| | | | | | 2011) | | |
| ZNF526 | Missense | HMZ | Inherited | Neuro | Mild non-syndromic | None | Novel Noonan |
| | | | | | ID (Najmabadi et al. | | like phenotype |
| | | | | | 2011) | | GDD |
| WDR45B | Nonsense | HMZ | Inherited | Neuro | ID and microcephaly | Primary | Epilepsy |
| (WDR45L) | | | | | (Najmabadi et al, | microcephaly | White matter |
| | | | | | 2011) | | changes, brain |
| | | | | | | | atrophy, |
| | | | | | | | hypoplastic |
| | | | | | | | corpus callosum |
| WDR81 | Nonsense | HMZ | Inherited | Neuro | Cerebellar | Cerebellar | Normal corpus |
| | | | | | ataxia, Mental | hypoplasia | callosum |
| | | | | | retardation, and | | Prenatal onset |
| | | | | | Dysequilibrium | | complicated by |
| | | | | | syndrome 2 | | neonatal death |

(*) Atypical case is defined as a case that has unusual clinical features, unusual mode of inheritance, a novel phenotype or lack of typical features.
DD: Dysmorphia-Dysplasia Panel,
GDD: Global Developmental Delay,
FTT: Failure to Thrive

Large scale genomic studies offer opportunities to improve the annotation of the human variome. This study, in which more than 2,300 well phenotyped human patients in a highly consanguineous population have been specifically tested for established disease genes, offered several advantages. First, the study was able to confirm genes that were only considered candidates because their candidacy was based on single mutations/families, so their status based on this study should be upgraded in the Online Mendelian Inheritance in Man (OMIM) database as such (e.g., ARL14EP, ZNF526, WDR45B, and WDR81). Second, the study added 446 novel disease alleles from a total of 795 variants, the largest to be reported in a single study. Third, the very large number of variants identified in the course of this study represented an unprecedented resource on the Arab variome (nearly all patients in this study were Arab in ethnicity), and this will be invaluable to the interpretation of clinical molecular genetic tests on Mendelian genes in Arab patients since it will help address the uncertainty surrounding the identification of many Arab-specific or Arab-enriched variants. Fourth, the high degree of consanguinity allowed the study to observe many variants in homozygosity as a result of autozygosity. This is particularly helpful when these variants were previously reported as disease-causing because observing them in the homozygous state at a relatively high population frequency strongly argues against their purported disease link. Furthermore, the finding of previously reported disease genes that harbor apparently inactivating mutations in the homozygous state at a relatively high frequency and in patients who lack the purported phenotype challenges their listing as disease genes (e.g., CACNA1F, MYH8, and PRX1) although it is acknowledged they have a potential role of such confounding factors as reduced penetrance.

The above method was initially limited to genes that were very likely to be disease-causing in a Mendelian context (based on the best available evidence) in order to eliminate the uncertainty surrounding the finding of variants in genes not known to be linked to human diseases. The study mainly included genes whose pathogenicity was supported by the presence of two pathogenic alleles. However, exceptions were made for genes with a single reported mutation but which were further supported by compelling mouse data or positional mapping data. This is important because it must be acknowledged that clinical WGS/WES currently appears to saddle the divide between clinical care and research.

If the Mendeliome assay is negative, it may be easier to prepare the patient for the possibility of identifying a novel genetic cause by WGS/WES that requires confirmation in a research setting. Unlike currently available gene panels, the present method seeks to be as inclusive as possible to minimize the challenge of atypical cases. For example, a gene for myopia presenting with ectopia lentis would still be identified because virtually every gene known to present with a prominent eye phenotype was included in the vision panel. In fact, the present analysis showed that only 3% (62/2,357) of cases may have been missed because the gene was not included in the right panel, and even this limitation can be addressed through a spike-in design. Such a broad and inclusive design was particularly helpful in disease categories that are characterized by a very high rate of heterogeneity. In addition to the vision panel, the high rate of atypical cases identified by the dysmorphology/dysplasia, neurology and immunology panels are also noted, although such cases were encountered in nearly all the panels.

Patients with various hereditary disorders most often are referred to the medical geneticist either through their primary care provider or through a medical subspecialist who attended to most prominent clinical presentation (i.e., neurological, ophthalmology, skin, renal, hematological, etc.). Therefore, the present symptom/sign based gene panels, collectively known as “The Mendeliome”, were designed in a way that simulates the way these patients present in clinical practice to the respective specialty.

Mendelian disorders are defined as hereditary disorders caused by a single autosomal or X-linked gene. The OMIM database, which currently contains about 4,300 monogenic disorders associated with known Molecular defects, represents the most comprehensive source of such information on monogenic disorders. Therefore, it was used as the primary source for gene identification. However, it was manually curated to ensure that only genes with confirmed links to disease are included. It was also supplemented with additional data from PubMed, Genetic Testing Registry (GTR), and gene tests. As such, the above 13 gene panels, which cover the spectrum of pediatric and adult clinical genetic medicine, were constructed. Within each panel, genes were sorted based on the most prominent sign/symptom with which they are most likely to be associated upon presentation to clinical care. This presentation may help the referring clinician, and without requiring sophisticated knowledge about these genes, decide on the appropriateness of genetic testing using these gene panels. Since many genetic disorders are as likely to present to several medical specialties, the present method allows for redundancy between the different panels (average 15%) such that a gene may be present in more than one panel.

3,070 genes covering over 4,000 Mendelian disorders (as annotated by OMIM up to August of 2013) were used as a basis for the design and synthesis of the highly multiplexed gene panels using Ion AmpliSeq Designer software (produced by Life Technologies of California). Tables 1-3 display the list of genes, their corresponding panels, information about the used transcripts, physical positions, and number of exons. From these 3,070 genes, there are 2,826 genes already listed in the genetic testing registry (GTR). Thirteen panels encompassing nearly all of the OMIM genes were defined broadly based upon clinical disciplines with some redundancy in gene content of individual panels. Primer design was based upon generating amplicons with an average length of 200 bp providing 90% minimum coverage of the coding DNA sequence (CDS) and on average 10 bp flanking regions of associated exons. Following this, in silico design coverage was assessed for compliance with design criteria and manual processes applied on a gene by gene basis to ensure adequate coverage and resolve factors such as 3′-SNPs that could impact primer efficiency. Primers for each panel were then synthesized and pooled into two multiplex reactions based upon polymerase chain reaction (PCR) compatibility minimizing likelihood of primer-primer interactions. Following this, synthesis primer pools were tested for coverage, recommended multiplexing and other quality control (QC) metrics to ensure specifications were met. Panels ranged from 96-758 gene with >90% coverage in 97-100% of genes in each panel.

Ten nanograms each of all DNA samples were treated to obtain the Ion Proton AmpliSeq library for one of the thirteen gene panels, as appropriate. DNA was amplified with 10-15 amplification cycles. PCR pools for each sample were combined and subjected to primer digestion with a FuPa reagent. Pooled amplicons were then ligated with universal adapters. After purification, libraries were quantitated by qPCR and normalized to 100 pM. Normalized libraries were barcoded (ligated with 24 different Ion Xpress Barcode adapters) and pooled in equal ratios for emulsion PCR (ePCR) on an Ion OneTouch System. Following ePCR, templated Ion Sphere particles were enriched using the Ion OneTouch ES. Both ePCR and enrichment procedures followed the manufacturer's instructions. The template-positive Ion PI Ion Sphere particles were processed for sequencing on the Ion Proton instrument.

The data of each run has been analyzed through a multistep pipeline. In the first step of this pipeline, the quality of the reads were verified and regions of the reads with low quality (less than 20) were trimmed out before alignment. The runs with low yield after this quality check were excluded. In the second step, the reads were aligned to the reference hg19 sequence. The observed depth after alignment ranges from 162X (for the neurology panel including 758 genes) to 840X (for the renal panel including 96 genes). In the third step, the aligned reads were processed for variant calling. In the subsequent step, the variants were annotated using public knowledge databases as well as in-house variants databases. The in-house databases include collections of disease-causing variants published by different Saudi teams and aggregation of the variants produced by the samples in this study.

In the final step of the pipeline, the non-relevant variants were filtered out based on their functional characteristics and their abundance in the datasets. Variants that are less likely to play a functional role (intronic and synonymous) and variants that were present in population databases (e.g., in the 1000Genome database with MAF>1%) were filtered out. Furthermore, variants that were frequent in the in-house database were also filtered out; a variant with more than 20 occurrences was considered frequent. The cutoff of 20 occurrences was selected on test data to assure 100% sensitivity. An individual base quality of 100 (using Phred-like score) was also selected to exclude low confidence variants. The few remaining variants were then analyzed based on relevance of gene to phenotype, zygosity (when indicated), and SIFT and PolyPhen scores (for missense variants). Table 16 below shows the efficiency of the filtering strategy. Table 16 shows that the subsequent filtering steps lead to a short list of variants to be examined by domain experts. In this table, and as expected, the larger the panel, the larger the list. It is also important to note that more samples included in the in-house database leads to more filtration power and makes the list even shorter. Ultimately, the recognized causal variant was identified as pathogenic or likely pathogenic as defined by the recent American College of Medical Genetics and Genomics (ACMG) guidelines, and the extensive variant data obtained by sequencing thousands of ethnically comparable patients (Saudis) was helpful in applying population frequency as a reliable criterion for pathogenicity in this study.

TABLE 16
Filtering Results Over All Variant Files

| | | | Public | SGP | | |
|---|---|---|---|---|---|---|
| | | Functional | Pop. | Pop. | | Zy- |
| Panel | Input | Sites | DBs | DB | Quality | gosity |
| Cardiovascular | 746 | 338 | 76 | 26 | 9 | 2 |
| Deafness | 828 | 257 | 63 | 50 | 17 | 6 |
| Dermatology | 1113 | 271 | 71 | 41 | 17 | 5 |
| Dysmorphology - | 1529 | 369 | 80 | 43 | 15 | 2 |
| Dysplasia | | | | | | |
| Endocrinology | 1129 | 326 | 61 | 42 | 19 | 5 |
| Gastroenterology | 362 | 190 | 60 | 20 | 6 | 1 |
| Hematology | 1474 | 324 | 79 | 39 | 18 | 3 |
| Inborn Errors of | 1955 | 571 | 94 | 54 | 24 | 4 |
| Metabolism | | | | | | |
| Neurology | 2885 | 718 | 158 | 87 | 29 | 4 |
| PID | 633 | 309 | 111 | 22 | 6 | 1 |
| Pulmonology | 723 | 230 | 74 | 39 | 21 | 3 |
| Renal | 507 | 132 | 35 | 21 | 7 | 1 |
| Vision | 906 | 341 | 75 | 51 | 17 | 3 |
| Total (Averages) | 1138 | 337 | 80 | 41 | 16 | 3 |

Given that the Mendeliome assay is inherently limited to established disease genes and will miss cases caused by large structural variants, 213 eases that are negative by the Mendeliome assay were randomly selected and processed using molecular karyotyping. CytoScan HD arrays were used for the majority of the patients. This array platform contains 2.6 million markers for copy number variation (CNV) detection, of which 750,000 are genotype SNPs and 1.9 million are nonpolymorphic probes, for whole genome coverage. Briefly, 250 ng of genomic DNA was digested with the restriction enzyme NspI and then ligated to an adapter, followed by polymerase chain reaction (PCR) amplification using a single pair of primers that recognized the adapter sequence. The PCR products were run on a 2% Tris-borate-EDTA (TBE) gel to confirm that the majority of products were between 150 and 2,000 bp in length.

To obtain a sufficient quantity of PCR product for further analysis, all products from each sample were combined and purified using magnetic beads. The purified PCR products were fragmented using DNase I and visualized on a 4% TBE agarose gel to confirm that the fragment sizes ranged from 25 to 125 bp. The fragmented PCR products were subsequently end-labeled with biotin and hybridized to the array. Arrays were then washed and stained, and then scanned and analyzed. The hidden Markov model was used to determine the copy-number states and their breakpoints. Thresholds of log₂ ratio ≧0.58 and ≦−1 were used to categorize altered regions as CNV gains (amplification) and copy-number losses (deletions), respectively.

To minimize the detection of false-positive CNVs arising due to inherent microarray noise, only alterations that involved at least 50 consecutive probes and that were at least 500 kb in size were used to categorize altered regions as CNV gains (amplification), whereas those at least 200 kb in size were used to categorize copy-number losses (deletions). The CNVs detected in the patients were then evaluated based on the ACMG standards and guidelines.

The genic content in the CNV interval of all the patients who had a molecular karyotype performed was taken into consideration by seeking recent publications to compare breakpoints, phenotypes, and different sizes of CNVs that overlapped. To exclude aberrations representing common benign CNVs, all the identified CNVs were compared with those reported in the Database of Genomic Variants and those reported in the in-house database for individuals who have been classified as normal.

De novo CNVs that met the size cutoff of 200 kb for deletions and 500 kb for duplications (based on the laboratory's consideration of the performance characteristics of the assay used) and were not found in either parent were classified as pathogenic. However, this does not eliminate the possibility that pathogenic CNVs exhibiting incomplete penetrance or variable expressivity can be present in an unaffected parent.

The remaining 178 were processed using WES. One hundred nanograms of each DNA sample was treated to obtain the Ion Proton AmpliSeq library. Briefly, DNA was amplified in twelve separate wells with 10 amplification cycles. All twelve PCR pools were combined in one well and subjected to primer digestion performing incubation with FuPa reagent. Amplified exome targets were ligated with Ion P1 and Ion Xpress Barcode adapters. Following this, purification libraries were quantified using qPCR. The prepared exome library was further used for emulsion PCR and templated Ion Sphere particles were enriched using Ion OneTouch ES, both procedures following the manufacturer's instructions. The template-positive Ion PI Ion Sphere particles were processed for sequencing on the Ion Proton instrument. Approximately 15-17 Gb of sequence was generated per sequencing run.

It is to be understood that the present invention is not limited to the embodiments described above, but encompasses any and all embodiments within the scope of the following claims.

I claim:

1. A method for diagnosing cardiovascular disease in a patent, comprising the steps of:
obtaining a sample from a patient;
assaying the sample to determine the presence of at least one genetic marker; and
diagnosing the patient with a cardiovascular disease if the at least one genetic marker is detected, wherein the at least one genetic marker is selected from the group consisting of TTR, MYPN, TTN, COL4A3, KCNH2, SMAD4, NOTCH1, ANK2, PKP2, LDB3, MYH6, MYBPC3, SCN5A, MYL3, CACNA1C, DMD, BAG3, EHMT1, DSG2, ABCC9, KCNE2, RYR2, TTN, TTN-AS1, VCL, SOS1, ANKRD1, ACTN2, DSP, FBN1, CHD7 and combinations thereof.
